(19) United States
(12) Patent Application Publication
(10) Pub. No.: US 2026/0076585 A1
(43) Pub. Date: Mar. 19, 2026

(54) METHODS AND SYSTEMS FOR ANALYZING AIRWAY EVENTS

(71) Applicant: Texas Medical Center, Houston, TX (US)

(72) Inventors: Onur Kilic, College Station, TX (US); Meagan Rochelle Pitcher, Houston, TX (US); Brittain Ancel Paul Cross, Houston, TX (US)

(21) Appl. No.: 18/870,906

(22) PCT Filed: Jun. 1, 2023
(86) PCT No.: PCT/US2023/024165

§ 371 Date: Dec. 2, 2024

Related U.S. Application Data
(60) Provisional application No. 63/365,835, filed on Jun. 3, 2022.

(51) Int. Cl. A61B 5/08; A61B 5/00; A61B 5/0205; A61B 5/024; A61B 5/087; A61B 5/11; A61B 5/113; A61B 5/1455; A61B 5/318; A61B 5/369; A61B 5/389; A61B 5/398

(52) U.S. Cl. CPC A61B 5/0803; A61B 5/0205; A61B 5/024; A61B 5/0816; A61B 5/087; A61B 5/1118; A61B 5/113; A61B 5/14551; A61B 5/318; A61B 5/369; A61B 5/389; A61B 5/398; A61B 5/4803; A61B 5/4818; A61B 5/6814; A61B 5/6822; A61B 5/6823; A61B 5/7246; A61B 5/7267; A61B 5/742; A61B 2560/0223; A61B 2562/0204; A61B 2562/0219

(57) ABSTRACT

The present disclosure is related to the field of detecting, localizing, and classifying an airway event in a subject. The method further includes a) removably securing one or more primary external sensors of the first plurality of external sensors to the head, face, neck, and/or upper torso of the subject prior to obtaining the vocalization dataset and/or b) positioning one or more secondary external sensors of the first plurality of external sensors at an optimal distance from the head, face, neck, and/or upper torso of the subject prior to obtaining the vocalization dataset; wherein the one or more secondary external sensors are not in direct contact with the subject.

### CROSS-REFERENCE TO RELATED APPLICATIONS

The present application claims the benefit of and priority to U.S. Patent Application No. 63/365,835, filed Jun. 3, 2022, the entire disclosure of which is incorporated herein in its entirety.

### FIELD OF THE INVENTION

The present disclosure is related to the field of detecting, localizing, and classifying an airway event in a subject.

### BACKGROUND OF THE INVENTION

Obstructive sleep apnea (OSA) is a common disorder characterized by repetitive collapse or narrowing of the upper airway passages during sleep that impairs ventilation and disrupts sleep. Factors that contribute to upper airway collapse include reduced upper-airway dilator muscle activity during sleep, specific upper-airway anatomical features, decreased end-expiratory lung volume, ventilatory control instability, and sleep-state instability. A collapse or narrowing of the airway passages during sleep may result in total or near total cessation of breathing or a partial reduction of ventilation.

Persons with OSA have a 30% higher risk of heart attack or death than those unaffected. Over time, OSA constitutes an independent risk factor for several diseases, including systemic hypertension, cardiovascular disease, stroke, and abnormal glucose metabolism. The estimated prevalence has increased in epidemiological studies over time and is in the range from 9% to 37% in men and from 4 to 50% in women. Sleep apnea requires expensive diagnostic and intervention paradigms, which are only available for a limited number of patients due to unavailability of sleep laboratories in each hospital. Hence, many patients with sleep apnea remain undiagnosed and untreated.

Thus, there is a need for new methods and systems that can facilitate the diagnosis of snoring, hypopnea, and apnea such that more patients can be treated without undergoing expensive and labor-intensive full night polysomnography.

### SUMMARY OF THE INVENTION

The present invention provides methods and systems for detecting, localizing, and classifying a subject's airway. In some embodiments, the airway is analyzed to diagnose a sleep disorder or a breathing event.

In a first aspect the invention provides a method of analyzing an airway of a subject, where the method includes a) obtaining a vocalization dataset via a first plurality of external sensors based on the subject articulating a plurality of calibration sounds; b) generating a mapped vocalization dataset by mapping the vocalization dataset to the airway of the subject or a portion thereof; c) obtaining an airway dataset of the airway of the subject via the first plurality of external sensors and/or a second plurality of external sensors; d) identifying a breathing event based on the airway dataset; and e) localizing the breathing event to one or more locations of the airway or the portion thereof using the airway dataset and the mapped vocalization dataset; wherein steps a) through e) are performed in any order of sequence. For example, step d) may occur prior to step e), or vice versa.

In some embodiments, the method further includes a) removably securing one or more primary external sensors of the first plurality of external sensors to the head, face, neck, and/or upper torso of the subject prior to obtaining the vocalization dataset and/or b) positioning one or more secondary external sensors of the first plurality of external sensors at an optimal distance from the head, face, neck, and/or upper torso of the subject prior to obtaining the vocalization dataset; wherein the one or more secondary external sensors are not in direct contact with the subject.

In some embodiments, the optimal distance is from about 1 cm to about 500 cm.

In some embodiments, the vocalization dataset includes tissue-borne sound data of a plurality of frequency bands for each calibration sound articulated by the subject.

In some embodiments, the calibration sounds comprise speech-based consonant sounds and/or non-speech-based consonant-like sounds produced by the mouth and/or airway of the subject.

In some embodiments, mapping the vocalization dataset includes measuring i) an amplitude difference and ii) a time difference corresponding to each calibration sound and wherein i) and ii) are measured by one or more of the external sensors in relation to a reference sensor.

In some embodiments, the method further includes correlating each amplitude difference and time difference measurement to a location of the one or more locations, based on the corresponding calibration sound, so as to generate a calibration dataset.

In some embodiments, localizing the breathing event includes a) obtaining, with the one or more plurality of external sensors, i) an amplitude difference measurement and ii) a time difference measurement for the breathing event; and b) comparing the amplitude difference measurement and the time difference measurement for the breathing event to the amplitude difference measurement and the time difference measurement of the calibration dataset; wherein i) and ii) are measured in relation to the reference sensor for a prescribed duration.

In some embodiments, the prescribed duration is a time interval of from about 10 seconds to 1 hour.

In some embodiments, the breathing event is produced by the subject during sleep.

In some embodiments, the breathing event includes an airway collapse event, a sleep apnea event, a partial airway collapse event, an apneic event, a hypoapneic event, a snore event, an upper airway occlusion event, a cessation of breathing, a respiratory disturbance event, ventilatory instability, normal breathing, a change in airflow, or any combination thereof.

In some embodiments, the one or more locations of the airway, or the portion thereof, includes a velum, an oropharynx, a hypopharynx, a tongue, and/or an epiglottis.

In some embodiments, the first and/or second pluralities of external sensors include from 2 to 10 external sensors.

In some embodiments, the method further includes removably securing the first plurality of external sensors on opposite sides of the subject's head or neck.

In some embodiments, the method further includes removably securing the first plurality of external sensors on the same side of the subject's head or neck.

In some embodiments, each of the first plurality of external sensors and/or the second plurality of external sensors includes a microphone and/or an accelerometer. In some embodiments, each of the first plurality of external sensors and/or the second plurality of external sensors further includes a sound emitting unit.

In some embodiments, the vocalization dataset is obtained while the subject is awake. In some embodiments, the first plurality of external sensors are located at a first plurality of positions of a head, face, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained.

In some embodiments, the airway dataset further includes one or more sounds associated with an airway condition comprising anaphylaxis, upper airway resistance syndrome, throat cancer, head cancer, neck cancer, chronic obstructive pulmonary disease, stridor, speech language disorders, a speech impediment, an accent, an infection, inflammation, an airway narrowing, or a laryngeal web.

In some embodiments, the method further includes a) teaching a machine learning software to identify a breathing event and/or classify a breathing event using a reference dataset; and b) presenting the airway dataset having one or more breathing events to the machine learning software; wherein the machine learning software compares the one or more breathing events to the reference dataset and to identify the breathing event and/or to classify the breathing event as a normal or abnormal breathing event.

In some embodiments, the method includes classifying the breathing event as a lateral collapse, an anterior-to-posterior collapse, or a concentric collapse.

In some embodiments, obtaining the airway dataset includes determining the location of one or more sounds in the airway using beamforming or reference mapping.

In some embodiments, the first plurality of external sensors is removably secured to the forehead of the subject, a temple of the subject, a cheekbone of the subject, adjacent to a nose of the subject, a mastoid of the subject, a mandible of the subject, and/or a throat of the subject.

In some embodiments, the plurality of frequency bands span a frequency range of from about 20 Hz to about 20 kHz. In some embodiments, the plurality of frequency bands span a frequency range of from about 100 Hz to about 5 kHz. In some embodiments, the plurality of frequency bands includes from 2 to 1,000 frequency bands. In some embodiments, each frequency band includes a bandwidth of from about 5 Hz to about 2500 Hz.

In some embodiments, the method further includes determining one or more metric(s) including Apnea-Hypopnea Index (AHI), blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, and/or actigraphy.

In another aspect, the invention provides an airway analysis system having a) a first plurality of external sensors; b) a processor in operative communication with the first plurality of external sensors; and c) a memory unit storing instructions that, when executed by the processor, cause the system to perform a plurality of operations including: i) obtaining a vocalization dataset via the first plurality of external sensors based on the subject articulating a plurality of calibration sounds; ii) generating a mapped vocalization dataset by mapping the vocalization dataset to the airway of the subject or a portion thereof; iii) obtaining an airway dataset of the airway of the subject via the first plurality of external sensors and/or a second plurality of external sensors; iv) identifying a breathing event based on the airway dataset; and v) localizing the breathing event to one or more locations of the airway or the portion thereof using the airway dataset and the mapped vocalization dataset; wherein steps i) through v) are performed in any order of sequence. For example, step v) may occur prior to step iv), or vice versa.

In some embodiments, the system further includes a) one or more primary external sensors of the first plurality of external sensors configured to be removably secured to the head, face, neck, and/or upper torso of the subject and/or b) one or more secondary external sensors of the first plurality of external sensors configured to be positioned at an optimal distance from the head, face, neck, and/or upper torso of the subject, wherein the one or more secondary external sensors are configured to not directly contact the subject.

In some embodiments, the optimal distance is from about 1 cm to about 500 cm.

In some embodiments, the first plurality of external sensors and/or the second plurality of external sensors are configured to measure tissue-borne sound for a given location of an anatomical area of the subject when the subject articulates a calibration sound.

In some embodiments, the system further includes a computing device. In some embodiments, the computing device includes a machine learning program configured to identify and/or classify the breathing event, and correlate the breathing event to a location of the corresponding one or more locations of the airway of the subject, as a normal breathing event or an abnormal breathing event.

In some embodiments, the first plurality of external sensors includes from 2 to 10 external sensors. In some embodiments, each of the first and/or second plurality of external sensors includes a microphone and/or an accelerometer. In some embodiments, each of the first and/or second plurality of external sensors further includes a sound emitting unit. In some embodiments, the first and/or second plurality of external sensors are electrically connected to a sound recorder.

In some embodiments, the instructions, when executed by the processor, cause the system to perform operations further including displaying the airway dataset on a digital display monitor and transmitting the airway dataset to a machine learning program.

In some embodiments, the machine learning program includes a plurality of algorithms configured to compare a training dataset to the airway dataset to classify one or more breathing events as normal or abnormal breathing events.

In some embodiments, the vocalization dataset includes tissue-borne sound data of a plurality of frequency bands for each calibration sound articulated by the subject.

In some embodiments, the calibration sounds include speech-based consonant sounds and/or non-speech-based consonant-like sounds produced by the mouth and/or airway of the subject.

In some embodiments, the operation of mapping the vocalization dataset includes measuring i) an amplitude difference and ii) a time difference corresponding to each calibration sound and wherein i) and ii) are measured by one or more external sensors of the at least one plurality of external sensors in relation to a reference sensor.

In some embodiments, the system is further configured to correlate each amplitude difference and time difference measurement to a location of the one or more locations, based on the corresponding calibration sound, so as to generate a calibration dataset.

In some embodiments, the breathing event is produced by the subject during sleep. In some embodiments, the breathing event includes an airway collapse event, a partial airway collapse event, an apneic event, a hypoapneic event, a snore event, an upper airway occlusion event, a cessation of breathing, a respiratory disturbance event, ventilatory instability, normal breathing, a change in airflow, or any combination thereof.

In some embodiments, localizing the breathing event to the one or more locations of the airway of the subject, or a portion thereof, includes: a) obtaining, with the first and/or second plurality of external sensors i) an amplitude difference measurement and ii) a time difference measurement for the breathing event; and b) comparing i) and ii) to the calibration dataset; wherein an amplitude difference measurement and a time difference measurement are measured in relation to a reference sensor for a prescribed duration.

In some embodiments, the prescribed duration is a time interval of from about 10 seconds to 1 hour.

In some embodiments, the one or more locations of the airway of the subject, or the portion thereof, includes a velum, an oropharynx, a tongue, and/or an epiglottis.

In some embodiments, the first plurality of external sensors are located at a first plurality of positions of a head, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained.

In some embodiments, the system is further configured to determine one or more metric(s) including Apnea-Hypopnea Index (AHI), blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, and/or actigraphy substantially in parallel to the plurality of operations.

In another aspect, the invention provides a method of analyzing an airway of a subject including a) removably securing a plurality of external sensors to the subject, b) recording a vocalization dataset, c) recording an airway dataset, and d) comparing the airway dataset to the vocalization dataset, wherein recording the vocalization dataset includes recording the subject articulating a plurality of calibration sounds.

### BRIEF DESCRIPTION OF THE DRAWINGS

The above and further features will be more clearly appreciated from the following detailed description when taken in conjunction with the accompanying drawings. The drawings however are for illustration purposes of exemplary embodiments only, not for limitation.

FIG. 1 is a flow chart showing an exemplary group of steps in the method herein disclosed to detect, analyze, and classify a breathing event during a subject's sleep.

FIG. 2 is a schematic drawing showing an example of an anatomical map used to correlate a calibration sound to a location in a subject's airway.

FIG. 3 is a schematic drawing showing a cross-sectional side view of an exemplary language-, accent-, and/or dialect-independent airway anatomical map. The locations in the airway where a calibration sound is generated are labeled by numbers.

FIG. 4 is a schematic drawing showing an exemplary processor, a plurality of sensors (e.g., microphones) and each sensor having a communication cable to operably communicate the sensor to the processor.

FIG. 5 is a schematic drawing showing an exemplary processor, a sensor operably communicated to the processor via a cable, and a plurality of input ports of the processor.

FIG. 6 is a schematic drawing of a computing device having a display monitor showing exemplary results localizing a breathing event to a location in the airway of a subject and classifying the breathing event.

FIG. 7 is a drawing showing primary sensor locations on a subject's face and/or neck (denoted by the numbers 1, 2, 3, 4, 5, and 6. Locations 7 and 8 are secondary sensor locations where sensors are optionally positioned to collect sound from the throat (location 7) or airborne sound (location 8).

FIG. 8A is a graph showing air-borne sound obtained using a microphone positioned close to the face of a subject. The graph shows two separate traces, a representative trace of the sound recorded when the subject vocalized the consonant p (light grey) and a representative trace of the sound recorded when the subject vocalized the consonant b (dark grey).

FIG. 8B is a graph showing air-borne sound obtained using a microphone positioned close to the face of a subject. The graph shows two separate traces, a representative trace of the sound recorded when the subject vocalized the consonant p (light grey) and a representative trace of the sound recorded when the subject vocalized the consonant b (dark grey).

FIG. 8C is a graph showing air-borne sound obtained using a microphone positioned close to the face of a subject. The graph shows two separate traces, a representative trace of the sound recorded when the subject vocalized the consonant p (light grey) and a representative trace of the sound recorded when the subject vocalized the consonant b (dark grey).

FIG. 9 is a screen capture image of an exemplary configuration of an airway analysis program including an airway map where multiple locations of the airway are identified by a circle marker and a plurality of operations available to a user.

FIG. 10 is a screen capture image of an embodiment of an airway analysis program showing a first and second breathing event in the airway of a subject identified by the herein disclosed methods and systems.

FIG. 11 is a screen capture image of an embodiment of an airway analysis program showing a first, a second, and a third breathing event in the airway of a subject identified by the herein disclosed methods and systems.

FIG. 12 is a flow chart showing a sequence of steps used to process raw audio to generate an interpretation of the data.

FIG. 13 is a schematic diagram showing an example computer and its components for use in detecting, localizing, analyzing, and classifying one or more breathing events.

FIG. 14 is a flow chart showing an exemplary sequence of steps for detecting, localizing, and classifying a breathing event.

### DETAILED DESCRIPTION OF THE INVENTION

Disclosed herein are exemplary embodiments of a method, and a system, for analyzing an airway of a subject. Accordingly, various embodiments described herein include a method to detect, localize, and classify one or more breathing events in an airway of a subject. Additionally, embodiments of a system to detect, localize, and classify one or more breathing events in an airway of a subject are herein described.

### Definitions

In order for the present invention to be more readily understood, certain terms are first defined below. Additional definitions may be found within the detailed description of the disclosure.

The singular forms “a,” “an,” and “the” include the plurals unless the context clearly dictates otherwise.

Unless specifically stated or obvious from context, as used herein, the term “or” is understood to be inclusive and covers both “or” and “and”.

The term “including” is used to mean “including but not limited to.” “Including” and “including but not limited to” are used interchangeably.

The terms “e.g.,” and “i.e.,” as used herein, are used merely by way of example, without limitation intended, and should not be construed as referring only those items explicitly enumerated in the specification.

The terms “one or more”, “at least one”, “more than one”, and the like are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

Conversely, the term “no more than” includes each value less than the stated value. For example, “no more than 100 containers” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 containers.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between.

Throughout this specification, the word “comprise,” or variations such as “comprises” or “comprising” will be understood to imply the inclusion of a stated integer (or components) or group of integers (or components), but not the exclusion of any other integer (or components) or group of integers (or components).

Unless specifically stated or obvious from context, as used herein, the term “about” is understood as within a range of normal tolerance in the art, for example within 2 standard deviations of the mean. “About” can be understood to be within 10%, 9%, 8%, 7%, 6%, 5%, 4%, 3%, 2%, 1%, 0.5%, 0.1%, 0.05%, 0.01%, or 0.001% of the stated value. Unless otherwise clear from the context, all numerical values provided herein are modified by the term “about”.

The terms “patient,” “subject,” and “individual” may be used interchangeably and refer to either a human or a non-human animal. These terms include mammals such as humans, primates, livestock animals (e.g., cows, pigs), companion animals (e.g., dogs, cats) and rodents (e.g., mice and rats).

The term “non-human mammal” means a mammal which is not a human and includes, but is not limited to, a mouse, rat, rabbit, pig, cow, sheep, goat, dog, primate, or other non-human mammals typically used in research. As used herein, “mammals” includes the foregoing non-human mammals and humans.

The term “consonant,” “consonant sound,” “consonant articulation,” “consonant-like,” or a variant thereof, as used herein, refers to a speech or non-speech sound produced in any spoken language, dialect, or accent. As used herein, “consonant,” “consonant sound,” “consonant articulation,” “consonant-like,” or a variant thereof refers to sound produced by a partial or complete obstruction of an air stream by any of various constrictions of the speech organs (e.g., the mouth and/or the airway).

The term “continuous,” or variations thereof, as used herein, will be understood to refer to a procedure conducted without interruption and with a repetition rate at time intervals ranging from fractions of a second up to, for example, 1, 2, 5, or 10 minutes or longer.

The term “substantially identical configuration,” as used in the context of sensor configuration or positioning on a subject's face, refers to minimal to no changes in sensor positioning or configuration between the sensor configuration during the collection of a first dataset (e.g., a vocalization dataset) and the sensor configuration during the collection of a second dataset. In general, “substantially identical configuration” refers to position or configuration changes where tolerances of no more than 5 mm in each dimension, preferably of no more than 1 mm, may be tolerated.

As used herein, the terms “vocalize,” “vocalization,” “articulation,” “articulate,” or “variations thereof” are to be used in a generic sense, referring to spoken word, speech tones, or any voice generated sound.

### Methods of Detection, Localization, and Classification of Airway Data

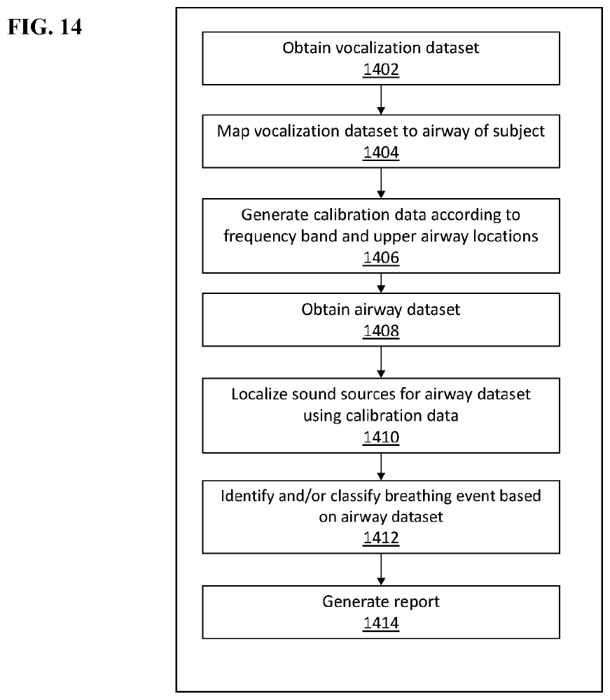

The invention includes methods of analyzing an airway of a subject. The methods are useful for identifying characteristic signatures of a subject's airway and analyzing the characteristic signatures in order to assess whether the subject's airway is in need of treatment due to abnormal breathing events. In general, the methods herein described include steps of obtaining a collection of signals (e.g., tissue-borne sounds) from a subject, herein referred to as a vocalization dataset (as in step 1402 of FIG. 14), mapping said signals to one or more locations of the airway of the subject (as seen in step 1404 of FIG. 14), generating a calibration dataset (as in step 1406 of FIG. 14), obtaining an airway dataset (as seen in step 1408 of FIG. 14), localizing sounds in the airway dataset using the calibration dataset and the mapped vocalization dataset (as seen in step 1410 of FIG. 14), identifying one or more breathing events and/or classifying the subject's breathing events (as seen in step 1412 of FIG. 14). In some embodiments, any one of steps 1402 through 1410 can be performed in any order of sequence. For example, step 1410 can be performed prior to step 1412, or vice versa. The method also includes the generation of a report of the results of the method (step 1414 of FIG. 14).

### Obtaining a Vocalization Dataset

In some embodiments, the method includes the use of at least a first plurality (e.g., from 2 to 10, e.g., 2, 3, 4, 5, 6, 7, 8, 9, or 10) of sensors, alternatively called “external sensors,” or “microphones” anywhere herein. In some embodiments, the method includes removably securing one or more primary external sensors of the first plurality of external sensors to the head, face, neck, and/or upper torso of the subject. In some embodiments, the method includes positioning one or more secondary external sensors, of the first plurality of external sensors, configured to not be in direct contact with the subject, at an optimal distance from the head, face, neck, and/or upper torso of the subject. In some embodiments, the secondary external sensors are part of a listening device. In some embodiments, the secondary external sensors are positioned at the bedside of a subject. In some embodiments, the optimal distance is from about 1 cm to about 500 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, about 100 cm, about 101 cm, about 102 cm, about 103 cm, about 104 cm, about 105 cm, about 106 cm, about 107 cm, about 108 cm, about 109 cm, about 110 cm, about 111 cm, about 112 cm, about 113 cm, about 114 cm, about 115 cm, about 116 cm, about 117 cm, about 118 cm, about 119 cm, about 120 cm, about 121 cm, about 122 cm, about 123 cm, about 124 cm, about 125 cm, about 126 cm, about 127 cm, about 128 cm, about 129 cm, about 130 cm, about 131 cm, about 132 cm, about 133 cm, about 134 cm, about 135 cm, about 136 cm, about 137 cm, about 138 cm, about 139 cm, about 140 cm, about 141 cm, about 142 cm, about 143 cm, about 144 cm, about 145 cm, about 146 cm, about 147 cm, about 148 cm, about 149 cm, about 150 cm, about 151 cm, about 152 cm, about 153 cm, about 154 cm, about 155 cm, about 156 cm, about 157 cm, about 158 cm, about 159 cm, about 160 cm, about 161 cm, about 162 cm, about 163 cm, about 164 cm, about 165 cm, about 166 cm, about 167 cm, about 168 cm, about 169 cm, about 170 cm, about 171 cm, about 172 cm, about 173 cm, about 174 cm, about 175 cm, about 176 cm, about 177 cm, about 178 cm, about 179 cm, about 180 cm, about 181 cm, about 182 cm, about 183 cm, about 184 cm, about 185 cm, about 186 cm, about 187 cm, about 188 cm, about 189 cm, about 190 cm, about 191 cm, about 192 cm, about 193 cm, about 194 cm, about 195 cm, about 196 cm, about 197 cm, about 198 cm, about 199 cm, about 200 cm, about 201 cm, about 202 cm, about 203 cm, about 204 cm, about 205 cm, about 206 cm, about 207 cm, about 208 cm, about 209 cm, about 210 cm, about 211 cm, about 212 cm, about 213 cm, about 214 cm, about 215 cm, about 216 cm, about 217 cm, about 218 cm, about 219 cm, about 220 cm, about 221 cm, about 222 cm, about 223 cm, about 224 cm, about 225 cm, about 226 cm, about 227 cm, about 228 cm, about 229 cm, about 230 cm, about 231 cm, about 232 cm, about 233 cm, about 234 cm, about 235 cm, about 236 cm, about 237 cm, about 238 cm, about 239 cm, about 240 cm, about 241 cm, about 242 cm, about 243 cm, about 244 cm, about 245 cm, about 246 cm, about 247 cm, about 248 cm, about 249 cm, about 250 cm, about 251 cm, about 252 cm, about 253 cm, about 254 cm, about 255 cm, about 256 cm, about 257 cm, about 258 cm, about 259 cm, about 260 cm, about 261 cm, about 262 cm, about 263 cm, about 264 cm, about 265 cm, about 266 cm, about 267 cm, about 268 cm, about 269 cm, about 270 cm, about 271 cm, about 272 cm, about 273 cm, about 274 cm, about 275 cm, about 276 cm, about 277 cm, about 278 cm, about 279 cm, about 280 cm, about 281 cm, about 282 cm, about 283 cm, about 284 cm, about 285 cm, about 286 cm, about 287 cm, about 288 cm, about 289 cm, about 290 cm, about 291 cm, about 292 cm, about 293 cm, about 294 cm, about 295 cm, about 296 cm, about 297 cm, about 298 cm, about 299 cm, about 300 cm, about 301 cm, about 302 cm, about 303 cm, about 304 cm, about 305 cm, about 306 cm, about 307 cm, about 308 cm, about 309 cm, about 310 cm, about 311 cm, about 312 cm, about 313 cm, about 314 cm, about 315 cm, about 316 cm, about 317 cm, about 318 cm, about 319 cm, about 320 cm, about 321 cm, about 322 cm, about 323 cm, about 324 cm, about 325 cm, about 326 cm, about 327 cm, about 328 cm, about 329 cm, about 330 cm, about 331 cm, about 332 cm, about 333 cm, about 334 cm, about 335 cm, about 336 cm, about 337 cm, about 338 cm, about 339 cm, about 340 cm, about 341 cm, about 342 cm, about 343 cm, about 344 cm, about 345 cm, about 346 cm, about 347 cm, about 348 cm, about 349 cm, about 350 cm, about 351 cm, about 352 cm, about 353 cm, about 354 cm, about 355 cm, about 356 cm, about 357 cm, about 358 cm, about 359 cm, about 360 cm, about 361 cm, about 362 cm, about 363 cm, about 364 cm, about 365 cm, about 366 cm, about 367 cm, about 368 cm, about 369 cm, about 370 cm, about 371 cm, about 372 cm, about 373 cm, about 374 cm, about 375 cm, about 376 cm, about 377 cm, about 378 cm, about 379 cm, about 380 cm, about 381 cm, about 382 cm, about 383 cm, about 384 cm, about 385 cm, about 386 cm, about 387 cm, about 388 cm, about 389 cm, about 390 cm, about 391 cm, about 392 cm, about 393 cm, about 394 cm, about 395 cm, about 396 cm, about 397 cm, about 398 cm, about 399 cm, about 400 cm, about 401 cm, about 402 cm, about 403 cm, about 404 cm, about 405 cm, about 406 cm, about 407 cm, about 408 cm, about 409 cm, about 410 cm, about 411 cm, about 412 cm, about 413 cm, about 414 cm, about 415 cm, about 416 cm, about 417 cm, about 418 cm, about 419 cm, about 420 cm, about 421 cm, about 422 cm, about 423 cm, about 424 cm, about 425 cm, about 426 cm, about 427 cm, about 428 cm, about 429 cm, about 430 cm, about 431 cm, about 432 cm, about 433 cm, about 434 cm, about 435 cm, about 436 cm, about 437 cm, about 438 cm, about 439 cm, about 440 cm, about 441 cm, about 442 cm, about 443 cm, about 444 cm, about 445 cm, about 446 cm, about 447 cm, about 448 cm, about 449 cm, about 450 cm, about 451 cm, about 452 cm, about 453 cm, about 454 cm, about 455 cm, about 456 cm, about 457 cm, about 458 cm, about 459 cm, about 460 cm, about 461 cm, about 462 cm, about 463 cm, about 464 cm, about 465 cm, about 466 cm, about 467 cm, about 468 cm, about 469 cm, about 470 cm, about 471 cm, about 472 cm, about 473 cm, about 474 cm, about 475 cm, about 476 cm, about 477 cm, about 478 cm, about 479 cm, about 480 cm, about 481 cm, about 482 cm, about 483 cm, about 484 cm, about 485 cm, about 486 cm, about 487 cm, about 488 cm, about 489 cm, about 490 cm, about 491 cm, about 492 cm, about 493 cm, about 494 cm, about 495 cm, about 496 cm, about 497 cm, about 498 cm, about 499 cm, or about 500 cm). In some embodiments, the sensors collect tissue-borne sound from the airway of the subject and convert the tissue-borne sound to electrical signals. In some embodiments, the sensors are removably secured to the face or neck of the subject using an adhesive, a tape, a sticker, or any other removable fastening item suitable for contacting the subject's skin and fastening the one or more sensors to the face or neck of the subject.

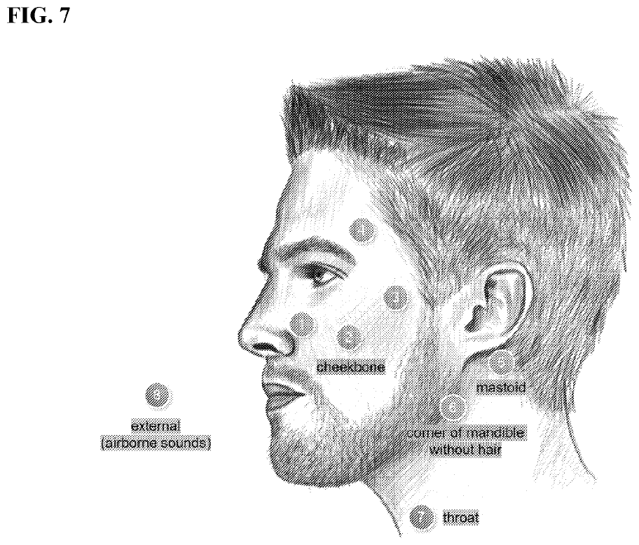

In some embodiments, the first plurality of external sensors are removably secured to opposite sides of the subject's face or neck. In some embodiments, the first plurality of external sensors are removably secured to the same side of the subject's face or neck (FIG. 7) (Image source for facial image of FIG. 7 is https://www.drawinghowtodraw.com/stepbystepdrawinglessons/wp-content/uploads/2017/09/how-to-draw-mans-male-face-from-the-side-profile-view-easy-stepbystep-drawing-tutorial-beginners.jpg). In some embodiments, sensors are positioned in regions of the face or neck substantially devoid of facial hair. For example, as seen in FIG. 7, primary sensor locations include those indicated by locations 1-6, and optional secondary sensor locations are indicated by positions 7 and 8. In some embodiments, the sensors are evenly distributed (e.g., with a substantially equal distance separating each sensor from the other sensors) on the face or neck of the subject. In some embodiments, the sensors are separated from each other by a distance of from about 10 mm to 100 mm (center-to-center) (e.g., about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, about 50 mm, about 51 mm, about 52 mm, about 53 mm, about 54 mm, about 55 mm, about 56 mm, about 57 mm, about 58 mm, about 59 mm, about 60 mm, about 61 mm, about 62 mm, about 63 mm, about 64 mm, about 65 mm, about 66 mm, about 67 mm, about 68 mm, about 69 mm, about 70 mm, about 71 mm, about 72 mm, about 73 mm, about 74 mm, about 75 mm, about 76 mm, about 77 mm, about 78 mm, about 79 mm, about 80 mm, about 81 mm, about 82 mm, about 83 mm, about 84 mm, about 85 mm, about 86 mm, about 87 mm, about 88 mm, about 89 mm, about 90 mm, about 91 mm, about 92 mm, about 93 mm, about 94 mm, about 95 mm, about 96 mm, about 97 mm, about 98 mm, about 99 mm, or about 100 mm). In some embodiments, the sensors are distributed in a regular configuration on the face or neck of the subject (e.g., a grid configuration, a triangular configuration, or a polygonal configuration. In some embodiments, the sensors are unevenly distributed (e.g., with a substantially unequal distance separating each sensor from the other sensors) on the face or neck of the subject. In some embodiments, the uneven distribution of the sensors provides increased sensitivity to the collection of airway data from an anatomical region of interest. In some embodiments, a plurality of sensors is removably secured to the face or neck of the subject. In some embodiments, collecting airway data with a plurality of sensors improves the signal-to-noise ratio of the airway data, thereby improving the quality of the airway data. In some embodiments, the plurality of sensors include the same number of sensors. In some embodiments, the plurality of sensors include different number of sensors. In some embodiments, the first plurality of sensors are removably secured to the forehead of the subject, a temple of the subject, a cheekbone of the subject, adjacent to a nose of the subject, a mastoid of the subject, a mandible of the subject, and/or a throat of the subject. In some embodiments, each of the first and/or the second plurality of external sensors include a microphone. In some embodiments, the first and/or second plurality of external sensors are microphones. In some embodiments, the first and/or second pluralities of sensors include a microphone and a sound emitting unit. In some embodiments, the sensors periodically produce short sounds (e.g., chirp) into the tissue of the face or neck of the subject for periodic corrections to the vocalization dataset based on small shifts in sensor position, or changes in temperature.

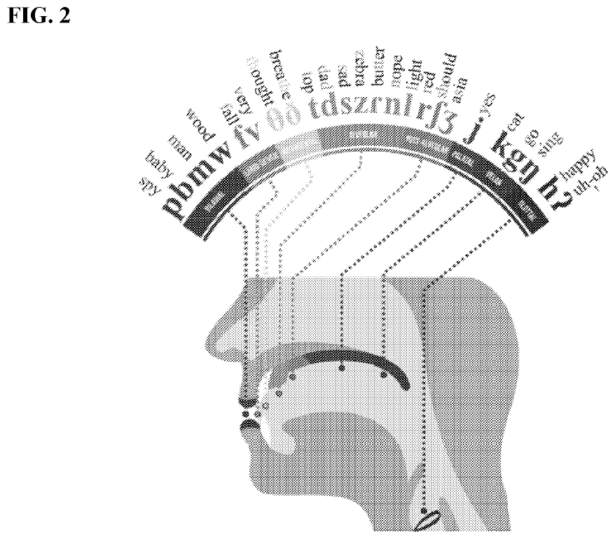

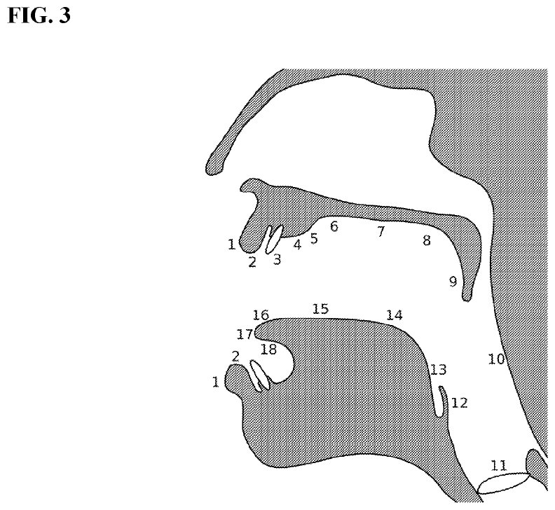

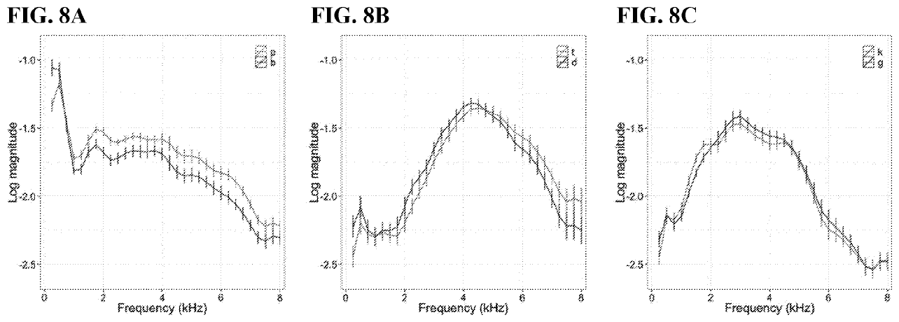

The method herein described includes the step of obtaining of a vocalization dataset. In some embodiments, the vocalization dataset is obtained from the subject while the subject is awake. In some embodiments, the vocalization dataset is obtained by recording, using at least a first plurality of sensors (e.g., the first and second plurality of external sensors), a plurality of calibration sounds articulated by the subject. In general, the location of articulation of a calibration sound is the location in the subject's airway where an obstruction or constriction of airflow takes place when producing the calibration sound during speech. In some embodiments, exemplary maps of locations of calibration sound articulation are used in the method of the invention (FIG. 2 and FIG. 3) (Image source for anatomical map image in FIG. 2 is http://people.fmarion.edu/llarsen/s203consonantimage.html) (Image source for anatomical map image in FIG. 3 is https://upload.wikimedia.org/wikipedia/commons/7/75/Places_of_articulation.svg). In some embodiments, the vocalization dataset may include speech-based sounds produced by a subject articulating one or more consonants. In some embodiments, the vocalization dataset may include non-speech-based consonant-like sounds produced by the subject (e.g., clicking sounds made with the tongue). A consonant-like sound may include, in some embodiments, any sound produced by the mouth or airway that at least partially constricts or obstructs airflow in the vocal tract/upper airway. In some embodiments, non-speech-based consonant-like sounds may also be used as calibration sounds of the vocalization dataset. In some embodiments, the vocalization dataset is obtained by recording, with at least a first plurality of sensors, a plurality of spoken words, containing consonant sounds originating from different regions of the subject's airway (FIG. 2), articulated by the subject. In some embodiments, each of the words in the plurality of spoken words includes one or more phonemes. In some embodiments, the vocalization dataset is stored in the memory unit of the processor unit described below (FIG. 2 and FIG. 8). In some embodiments, each articulation of a calibration sound or phrase is associated with sound data obtained via the plurality of sensors, including frequency, amplitude, and/or time difference (as described herein).

The vocalization dataset, in some embodiments, includes tissue-borne sound data collected in a plurality of frequency bands that span a frequency range of from about 20 Hz to about 20 kHz (e.g., from about 40 Hz to about 20 kHz, from about 60 Hz to about 15 kHz, from about 80 Hz to about 10 kHz, from about 100 Hz to about 5 kHz; e.g., about 100 Hz, about 200 Hz, about 300 Hz, about 400 Hz, about 500 Hz, about 600 Hz, about 700 Hz, about 800 Hz, about 900 Hz, about 1 kHz, about 1.1 kHz, about 1.2 kHz, about 1.3 kHz, about 1.4 kHz, about 1.5 kHz, about 1.6 kHz, about 1.7 kHz, about 1.8 kHz, about 1.9 kHz, about 2 kHz, about 2.1 kHz, about 2.2 kHz, about 2.3 kHz, about 2.4 kHz, about 2.5 kHz, about 2.6 kHz, about 2.7 kHz, about 2.8 kHz, about 2.9 kHz, about 2 kHz, about 3.1 kHz, about 3.2 kHz, about 3.3 kHz, about 3.4 kHz, about 3.5 kHz, about 3.6 kHz, about 3.7 kHz, about 3.8 kHz, about 3.9 kHz, about 2 kHz, about 4.1 kHz, about 4.2 kHz, about 4.3 kHz, about 4.4 kHz, about 4.5 kHz, about 4.6 kHz, about 4.7 kHz, about 4.8 kHz, about 4.9 kHz, or about 5 kHz). In some embodiments, the vocalization dataset is parsed into a plurality of frequency bands (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 200, 300, 400, 500, 600, 700, 800, 900, or 1000 frequency bands). In some embodiments, the vocalization dataset is parsed into a plurality of frequency bands that each have a bandwidth of from about 5 Hz to about 2.5 kHz (e.g., about 5 Hz, about 6 Hz, about 7 Hz, about 8 Hz, about 9 Hz, about 10 Hz, about 11 Hz, about 12 Hz, about 13 Hz, about 14 Hz, about 15 Hz, about 16 Hz, about 17 Hz, about 18 Hz, about 19 Hz, about 20 Hz, about 21 Hz, about 22 Hz, about 23 Hz, about 24 Hz, about 25 Hz, about 26 Hz, about 27 Hz, about 28 Hz, about 29 Hz, about 30 Hz, about 31 Hz, about 32 Hz, about 33 Hz, about 34 Hz, about 35 Hz, about 36 Hz, about 37 Hz, about 38 Hz, about 39 Hz, about 40 Hz, about 41 Hz, about 42 Hz, about 43 Hz, about 44 Hz, about 45 Hz, about 46 Hz, about 47 Hz, about 48 Hz, about 49 Hz, about 50 Hz, about 51 Hz, about 52 Hz, about 53 Hz, about 54 Hz, about 55 Hz, about 56 Hz, about 57 Hz, about 58 Hz, about 59 Hz, about 60 Hz, about 61 Hz, about 62 Hz, about 63 Hz, about 64 Hz, about 65 Hz, about 66 Hz, about 67 Hz, about 68 Hz, about 69 Hz, about 70 Hz, about 71 Hz, about 72 Hz, about 73 Hz, about 74 Hz, about 75 Hz, about 76 Hz, about 77 Hz, about 78 Hz, about 79 Hz, about 80 Hz, about 81 Hz, about 82 Hz, about 83 Hz, about 84 Hz, about 85 Hz, about 86 Hz, about 87 Hz, about 88 Hz, about 89 Hz, about 90 Hz, about 91 Hz, about 92 Hz, about 93 Hz, about 94 Hz, about 95 Hz, about 96 Hz, about 97 Hz, about 98 Hz, about 99 Hz, about 100 Hz, about 200 Hz, about 300 Hz, about 400 Hz, about 500 Hz, about 600 Hz, about 700 Hz, about 800 Hz, about 900 Hz, about 1 kHz, about 1.1 kHz, about 1.2 kHz, about 1.3 kHz, about 1.4 kHz, about 1.5 kHz, about 1.6 kHz, about 1.7 kHz, about 1.8 kHz, about 1.9 kHz, about 2 kHz, about 2.1 kHz, about 2.2 kHz, about 2.3 kHz, about 2.4 kHz, or about 2.5 kHz). In some embodiments, the vocalization dataset includes tissue-borne sound data of a plurality of frequency bands, as disclosed herein, for each calibration articulated by the subject (FIGS. 8A-8C) (Data source for graphs in FIGS. 8A-8C is Chodroff, E., Wilson, C., Burst spectrum as a cue for the stop voicing contrast in American English, The Journal of the Acoustical Society of America, 136, 2762 (2014).). For example, in some embodiments, the vocalization dataset includes the tissue-borne sound produced by a subject when pronouncing the letter “t” as in the word “top” (FIG. 2 and FIG. 8B). In some embodiments, the calibration sounds include speech-based consonant sounds and/or non-speech-based consonant-like sounds produced by the mouth and/or airway of the subject. Additionally, vocalization data for a calibration, as seen in FIGS. 8A-8C, may be analyzed in segments or in pre-determined frequency bands (e.g., from 100 Hz to 2 kHz, from 2 kHz to 4 kHz, from 4 kHz to 6 kHz, or from 6 kHz to 8 kHz).

In some embodiments, the vocalization dataset is obtained by recording calibration sounds at increasing levels of loudness. For example, a subject may start by vocalizing a “k” sound, followed by a louder “k” sound, etc. In some embodiments, recording calibration sounds at multiple levels of loudness provides the user the option to correct for a signal being too strong or too weak for some of the sensors.

### Mapping a Vocalization Dataset

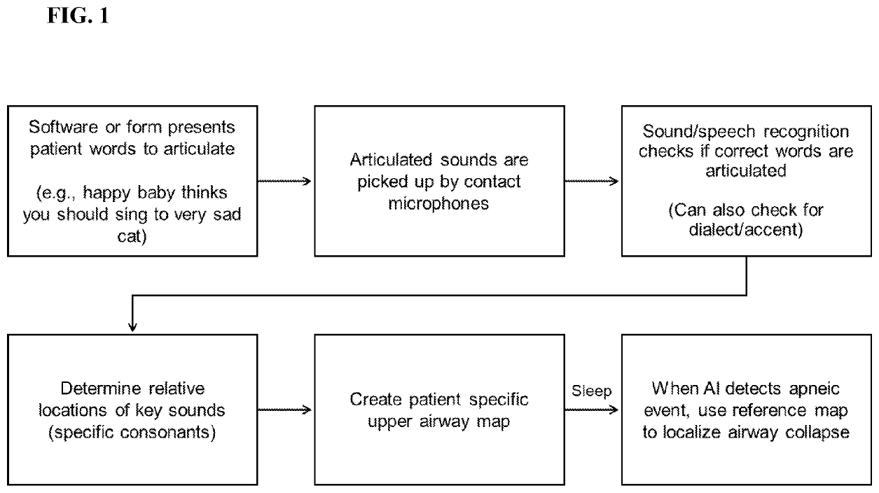

In some embodiments, the method includes generating a mapped vocalization dataset by mapping the vocalization dataset to the airway or a portion thereof of the subject. In some embodiments, the anatomical area of interest is the upper airway of the subject. In some embodiments, the vocalization dataset is mapped to one or more locations of the airway including the velum, the oropharynx, the hypopharynx, the tongue, and/or the epiglottis. In some embodiments, the mapping of the vocalization dataset includes correlating the sound pattern for each consonant articulated by a subject, upon reciting a standard phrase or set of words (e.g., “Uh-oh, hope you can think of a short sentence,” “Uh-oh, hide yourself octopus. I have sharp teeth,” “Hi, Pry, Fry, Thigh, Try, Shy, Yes, Cry, Uh-Oh, Bye,” or “happy baby thinks you should sing to very sad cat” as in FIG. 1), to a standardized map of vocalization sounds known in the art (FIG. 2). For example, as seen in FIG. 2, a “p” sound is known to originate at the lips of a subject, a “y” sound as in the word “yes” is known to originate from the palatal region of the upper airway, and the “h” sound in the word “uh-oh” is known to originate in the glottal region. In some embodiments, mapping the vocalization dataset, using the plurality of sensors, includes establishing one of the plurality of sensors as a reference sensor. In some embodiments, mapping the vocalization dataset includes measuring the amplitude, at each of the plurality of sensors, of a plurality of calibration sounds articulated by the subject and then determining the amplitude difference between the amplitude measured at each sensor and the amplitude measured by the reference sensor. In some embodiments, mapping the vocalization dataset also includes measuring the time from the articulation of each of a plurality of calibration sounds (e.g., consonant or consonant-like sounds) to the collection of each of the plurality of calibration sounds by each of the sensors. In some embodiments, the time difference between the time measured at each sensor and the time measured by the reference sensor is determined. In some embodiments, the amplitude difference and/or the time difference are the metrics extracted from the vocalization data to determine the location of each of the plurality of calibration sounds articulated by the subject. In some embodiments, correlating each amplitude difference and/or time difference measurement to a location of the one or more locations generates a calibration dataset. In some embodiments, the calibration dataset is determined for every subject specifically. In some embodiments, the calibration dataset is determined for every subject and for every airway analysis session for the same subject.

In general, since the method of obtaining a vocalization dataset, that can be further used as a calibration dataset for each subject, is collected de novo for every subject and for every study session, the method of mapping that subject's vocalization sounds and of localizing the subject's breathing events is agnostic to a subject's spoken language, dialect, or accent. The unique vocalization characteristics of a subject are captured and considered in the method of mapping subsequently obtained breathing event data.

### Obtaining an Airway Dataset

The method herein described includes the collection of an airway dataset. In some embodiments, the airway dataset is obtained from the airway or a portion thereof of the subject while the subject is awake or asleep. In some embodiments, the airway dataset is obtained from the subject while the subject is asleep. Additionally, in some embodiments, the airway dataset is obtained after the vocalization dataset is obtained. In some embodiments, the airway dataset is obtained immediately after the vocalization dataset is obtained. In some embodiments, the airway dataset is obtained by recording, using the first and/or second plurality of sensors, a plurality of sounds generated by the subject. In some embodiments, the airway dataset is obtained by recording, using the plurality of sensors, a plurality of sounds generated by the subject while the subject is asleep. In some embodiments, the first plurality of external sensors are located at a first plurality of positions of a head, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained. In some embodiments, the airway dataset is obtained by recording the plurality of sounds generated by the subject using a substantially identical configuration of the reversibly secured sensors as when the vocalization dataset and/or the calibration dataset was obtained. In some embodiments, the airway dataset is collected for a duration of from about 1 minute to about 8 hours (e.g., 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, 1 hour, 2 hours, 3 hours, 4 hours, 5 hours, 6 hours, 7 hours, or 8 hours). In some embodiments, the airway dataset includes one or more sounds associated with an airway condition comprising anaphylaxis, upper airway resistance syndrome, head and neck cancer (e.g., cancers of the mouth or throat), chronic obstructive pulmonary disease, stridor, a speech impediment, speech language disorders, an accent, an infection, inflammation, an airway narrowing, or a laryngeal web. In some embodiments, obtaining the airway dataset includes determining the location of one or more sounds in the airway, or a portion thereof, using beamforming, reference mapping methods, or variations thereof.

### Comparison Methods

In some embodiments, the localization of the vocalization dataset and/or the airway dataset includes beamforming or reference mapping. In some embodiments, reference mapping includes a calibration procedure using vocalization of calibration sounds to form a reference dataset and then comparing the reference dataset to a test dataset (e.g., an airway dataset during a subject's sleep). In some embodiments, for the comparison of datasets (e.g., reference dataset vs. test dataset) any one of the following similarity or dissimilarity measures (or any variation thereof) used conventionally in the field of Data Science may be utilized including: L2 norm Euclidean distance, Squared Euclidean Distance, L1 norm, City Block, Manhattan, taxicab distance, Canberra distance, L∞ norm, Chebyshev distance, maximum distance, Lp norm, Minkowski distance, Cosine distance, Pearson Correlation distance, Spearman correlation, Mahalanobis distance, Standardized Euclidian distance, Chi-square distance, Jensen-Shannon distance, Levenshtein distance, Hamming distance, Jaccard/Tanimoto distance, or Sørensen-Dice distance as seen in Harmouch M. “17 types of similarity and dissimilarity measures used in data science” Towards Data Science, 2021, https://towardsdatascience.com/17-types-of-similarity-and-dissimilarity-measures-used-in-data-science-3eb914d2681, herein incorporated by reference in its entirety.

### Identifying, Localizing, and Classifying a Breathing Event

The method of the present invention also includes the identification of a breathing event within the airway dataset. A breathing event may be characterized by a characteristic signature (e.g., amplitude and/or frequency) in a plurality of characteristic frequency bands. For example, a breathing event such as a normal breathing, chokes, and airway reopening sounds may be characterized by an acoustic signature in the frequency bands of 100 Hz to 500 Hz, 500 Hz to 1 kHz, 1 kHz to 2 kHz, and 2 kHz to 5 kHz. A breathing event, in some embodiments, includes an abnormal breathing event (e.g., an airway collapse event, a partial airway collapse event, an apneic event, a hypoapneic event, a snore event, an upper airway occlusion event, a cessation of breathing, a respiratory disturbance event, ventilatory instability, a cough, or any combination thereof) and a normal breathing event (e.g., normal breathing, a normal change in airflow, or any combination thereof). In some embodiments, the airway dataset is divided into a plurality of intervals of a prescribed duration. In some embodiments, the prescribed duration is a time interval of from about 10 seconds to about 1 hour (e.g., about 10 seconds, 11 seconds, 12 seconds, 13 seconds, 14 seconds, 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes, 30 minutes, 31 minutes, 32 minutes, 33 minutes, 34 minutes, 35 minutes, 36 minutes, 37 minutes, 38 minutes, 39 minutes, 40 minutes, 41 minutes, 42 minutes, 43 minutes, 44 minutes, 45 minutes, 46 minutes, 47 minutes, 48 minutes, 49 minutes, 50 minutes, 51 minutes, 52 minutes, 53 minutes, 54 minutes, 55 minutes, 56 minutes, 57 minutes, 58 minutes, 59 minutes, or 1 hour).

In some embodiments, the breathing event is localized to one or more locations of the airway, or a portion thereof, using the airway dataset and the mapped vocalization dataset. In some embodiments, one or more identified breathing events of the airway dataset are localized to a location in the airway of the subject using beamforming. For example, in some embodiments, one or more identified breathing events of the airway dataset are correlated to a location in the airway of the subject by 1) obtaining, with one or more sensors of the first and/or second plurality of sensors, an amplitude difference measurement and a time difference measurement for the breathing event, followed by 2) comparing the amplitude difference measurement and the time difference measurement for the breathing event to the amplitude difference measurement and the time difference measurement of the calibration dataset. In general, the amplitude and time differences are measured in relation to a reference sensor for a prescribed duration. In some embodiments, the comparison between the breathing event metrics (e.g., amplitude and time differences per frequency band) and the calibration dataset (e.g., amplitude and time difference per frequency band) is performed using any of the comparison methods (e.g., a similarity measure such as the Chi-Square method) herein disclosed.

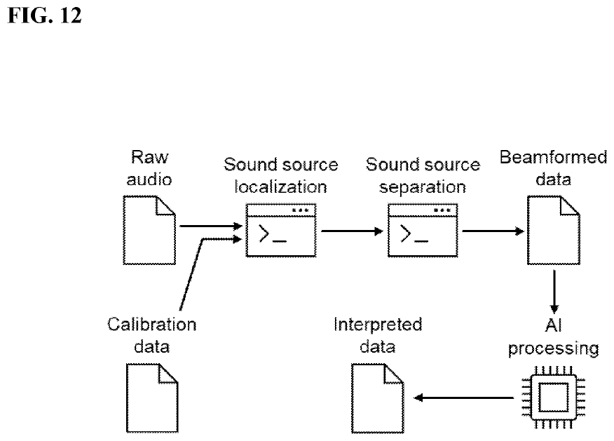

The method of the present invention includes the step of identifying the breathing event based on an acoustic signature in the frequency bands of 100 Hz to 500 Hz, 500 Hz to 1 kHz, 1 kHz to 2 kHz, and 2 kHz to 5 kHz. In some embodiments, the breathing event is identified before it is localized to a location of the airway. In some embodiments, the breathing event is identified after it is localized to a location of the airway (FIG. 12). In some embodiments, the breathing event is identified and localized to a location of the airway in a substantially simultaneous manner.

The method of the present invention includes the step of classifying the breathing event as a normal or an abnormal breathing event. In some embodiments, the breathing event is classified before it is correlated to a location of the airway. In some embodiments, the breathing event is classified after it is correlated to a location of the airway (FIG. 12). In some embodiments, the breathing event is classified and correlated to a location of the airway in a substantially simultaneous manner.

In some embodiments, the method of classification of one or more breathing events includes using and/or training a machine learning algorithm via a computing device (as described herein). In some embodiments, the method of classification of a breathing event as a normal or abnormal breathing event is based on identifying a characteristic acoustic signature as either normal or abnormal. In some embodiments, the machine learning algorithm is trained by inputting a training dataset into the machine learning algorithm. In some embodiments, the machine learning process is supervised. In some embodiments, the machine learning process is unsupervised. In some embodiments, the training dataset includes simulation data. In some embodiments, the machine learning algorithm is trained with finite-element multiphysics simulation data. In some embodiments, the training dataset includes clinical data from real patients. In some embodiments, the machine learning algorithm is a neural network. In some embodiments, the neural network is a deep neural network or a shallow neural network. In some embodiments, the deep neural network is a convolutional neural network. In some embodiments, the method of classification of one or more breathing events includes calculating a Mel-frequency cepstrum for each breathing event and then using a neural network to estimate the probability of the breathing event of being normal or abnormal.

In some embodiments, the machine learning algorithm classifies a breathing event (e.g., an apneic event due to an airway collapse) into a breathing event configuration. In some embodiments, the breathing event configuration includes a lateral collapse, an anterior-to-posterior collapse, or a concentric collapse. In some embodiments, the breathing event configuration includes an anterior-to-posterior collapse or a partial collapse. The method of classifying a breathing event using the machine learning algorithm, is useful for detecting and/or diagnosing a breathing event associated with natural sleep, snoring events, snore source location, an upper airway collapse, an upper airway obstruction, an upper airway occlusion, sleep apnea, upper airway resistance syndrome, chronic obstructive pulmonary disease, stridor, cessation of breathing, respiratory disturbance events, a stage of sleep, arousal threshold, ventilatory instability, arousal intensity, an airway anatomy feature in awake subjects, acute upper airway tissue changes, allergic reaction, anaphylaxis, head and neck cancer (e.g., cancers of the mouth or throat), pre-operative airway assessment for anesthesiology risk, a difficult airway in anesthesia, speech impediments, speech language disorders, accents, or any combination or variation thereof. In some embodiments, the breathing event location is used to train the machine learning algorithm. In some embodiments, training the machine learning algorithm with breathing event location data provides the machine learning algorithm with the capability to predict the location of breathing events in subsequently collected and/or analyzed airway datasets.

In some embodiments, the methods herein described can be performed in parallel to methods commonly used as the standard of care to determine metrics such as an Apnea-Hypopnea Index (AHI), blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, actigraphy, among others. In some embodiments, the methods herein described can be performed to predict metrics commonly used as the standard of care, such as an AHI blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, actigraphy, among others.

In some embodiments, a report and/or a graphical representation of the airway dataset is generated and presented to the sleep study specialist for treatment determination. In some embodiments, the machine learning program suggests a treatment for the subject experiencing one or more breathing events, based on the detected breathing event configuration. In some embodiments, the machine learning program can predict a response to a treatment. For example, a machine learning program, in some embodiments, is trained to predict that an anteroposterior epiglottic collapse is less probable to respond positively to maxillomandibular advancement (as seen in Zhou, N., et al., J. Clin. Sleep Med. (2002), 18(4):1073-1081, doi: 10.5664/jcsm.802, herein incorporated by reference in its entirety). In some embodiments, a recommendation of a treatment and/or further analysis is automatically generated.

### System for Airway Analysis

The invention herein described provides a system for airway analysis, including detecting, localizing and classifying airway data.

In some embodiments, the system includes at least one plurality (e.g., from 2 to 10, e.g., 2, 3, 4, 5, 6, 7, 8, 9, or 10) of external sensors. In some embodiments, the system includes a first plurality of external sensors configured to be removably secured to the head, face, neck, and/or upper torso of the subject. In some embodiments, the system includes a second plurality of external sensors configured to be positioned at an optimal distance from the head, face, neck, and/or upper torso of the subject. In some embodiments, the second plurality of sensors is configured to not directly contact the subject. In some embodiments, the optimal distance is from about 1 cm to about 500 cm (e.g., about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, about 30 cm, about 31 cm, about 32 cm, about 33 cm, about 34 cm, about 35 cm, about 36 cm, about 37 cm, about 38 cm, about 39 cm, about 40 cm, about 41 cm, about 42 cm, about 43 cm, about 44 cm, about 45 cm, about 46 cm, about 47 cm, about 48 cm, about 49 cm, about 50 cm, about 51 cm, about 52 cm, about 53 cm, about 54 cm, about 55 cm, about 56 cm, about 57 cm, about 58 cm, about 59 cm, about 60 cm, about 61 cm, about 62 cm, about 63 cm, about 64 cm, about 65 cm, about 66 cm, about 67 cm, about 68 cm, about 69 cm, about 70 cm, about 71 cm, about 72 cm, about 73 cm, about 74 cm, about 75 cm, about 76 cm, about 77 cm, about 78 cm, about 79 cm, about 80 cm, about 81 cm, about 82 cm, about 83 cm, about 84 cm, about 85 cm, about 86 cm, about 87 cm, about 88 cm, about 89 cm, about 90 cm, about 91 cm, about 92 cm, about 93 cm, about 94 cm, about 95 cm, about 96 cm, about 97 cm, about 98 cm, about 99 cm, or about 100 cm). In some embodiments, each of the first plurality of external sensors includes a microphone and/or an accelerometer. In some embodiments, the first plurality of external sensors are microphones. In some embodiments, the microphones are piezo-or electret-based. In some embodiments, the microphones are piezo-based microphones which can both collect and emit sound. In some embodiments, the microphones are configured to collect sound at an amplitude of from about 30 dB to about 90 dB (e.g., about 30 dB, about 31 dB, about 32 dB, about 33 dB, about 34 dB, about 35 dB, about 36 dB, about 37 dB, about 38 dB, about 39 dB, about 40 dB, about 41 dB, about 42 dB, about 43 dB, about 44 dB, about 45 dB, about 46 dB, about 47 dB, about 48 dB, about 49 dB, about 50 dB, about 51 dB, about 52 dB, about 53 dB, about 54 dB, about 55 dB, about 56 dB, about 57 dB, about 58 dB, about 59 dB, about 60 dB, about 61 dB, about 62 dB, about 63 dB, about 64 dB, about 65 dB, about 66 dB, about 67 dB, about 68 dB, about 69 dB, about 70 dB, about 71 dB, about 72 dB, about 73 dB, about 74 dB, about 75 dB, about 76 dB, about 77 dB, about 78 dB, about 79 dB, about 80 dB, about 81 dB, about 82 dB, about 83 dB, about 84 dB, about 85 dB, about 86 dB, about 87 dB, about 88 dB, about 89 dB, or about 90 dB) and at a frequency range from about 20 Hz to about 20 kHz (e.g., from about 40 Hz to about 20 kHz, from about 60 Hz to about 15 kHz, from about 80 Hz to about 10 kHz, or from about 100 Hz to about 5 kHz). In some embodiments, the microphones have a sensitivity of from about 5 m V/Pa to about 20 m V/Pa (e.g., about 5 m V/Pa, about 6 m V/Pa, about 7 m V/Pa, about 8 m V/Pa, about 9 m V/Pa, about 10 m V/Pa, about 11 m V/Pa, about 12 m V/Pa, about 13 m V/Pa, about 14 m V/Pa, about 15 m V/Pa, about 16 m V/Pa, about 17 m V/Pa, about 18 m V/Pa, about 19 m V/Pa, or about 20 mV/Pa). In some embodiments, the microphones have a dynamic range of from about 50 dB to about 100 dB (e.g., about 50 dB, about 51 dB, about 52 dB, about 53 dB, about 54 dB, about 55 dB, about 56 dB, about 57 dB, about 58 dB, about 59 dB, about 60 dB, about 61 dB, about 62 dB, about 63 dB, about 64 dB, about 65 dB, about 66 dB, about 67 dB, about 68 dB, about 69 dB, about 70 dB, about 71 dB, about 72 dB, about 73 dB, about 74 dB, about 75 dB, about 76 dB, about 77 dB, about 78 dB, about 79 dB, about 80 dB, about 81 dB, about 82 dB, about 83 dB, about 84 dB, about 85 dB, about 86 dB, about 87 dB, about 88 dB, about 89 dB, about 90 dB, about 91 dB, about 92 dB, about 93 dB, about 94 dB, about 95 dB, about 96 dB, about 97 dB, about 98 dB, about 99 dB, about 100 dB). In some embodiments, the first and/or second plurality of external sensors include a microphone and/or an accelerometer and a sound emitting unit. In some embodiments, the sound emitting unit is configured to emit sound at an amplitude of from about 30 dB to about 60 dB (e.g., about 30 dB, about 31 dB, about 32 dB, about 33 dB, about 34 dB, about 35 dB, about 36 dB, about 37 dB, about 38 dB, about 39 dB, about 40 dB, about 41 dB, about 42 dB, about 43 dB, about 44 dB, about 45 dB, about 46 dB, about 47 dB, about 48 dB, about 49 dB, about 50 dB, about 51 dB, about 52 dB, about 53 dB, about 54 dB, about 55 dB, about 56 dB, about 57 dB, about 58 dB, about 59 dB, or about 60 dB) and at a frequency range from about 1 kHz to about 50 kHz (e.g., about 1 kHz, about 2 kHz, about 3 kHz, about 4 kHz, about 5 kHz, about 6 kHz, about 7 kHz, about 8 kHz, about 9 kHz, about 10 kHz, about 11 kHz, about 12 kHz, about 13 kHz, about 14 kHz, about 15 kHz, about 16 kHz, about 17 kHz, about 18 kHz, about 19 kHz, about 20 kHz, about 21 kHz, about 22 kHz, about 23 kHz, about 24 kHz, about 25 kHz, about 26 kHz, about 27 kHz, about 28 kHz, about 29 kHz, about 30 kHz, about 31 kHz, about 32 kHz, about 33 kHz, about 34 kHz, about 35 kHz, about 36 kHz, about 37 kHz, about 38 kHz, about 39 kHz, about 40 kHz, about 41 kHz, about 42 kHz, about 43 kHz, about 44 kHz, about 45 kHz, about 46 kHz, about 47 kHz, about 48 kHz, about 49 kHz, about 50 kHz). In some embodiments, the sensors have a circular, square, rectangular, triangular, elliptical, star, or polygonal shape. In some embodiments, the sensors have a circular shape and a diameter of from about 10 mm to about 50 mm (e.g., about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, about 25 mm, about 26 mm, about 27 mm, about 28 mm, about 29 mm, about 30 mm, about 31 mm, about 32 mm, about 33 mm, about 34 mm, about 35 mm, about 36 mm, about 37 mm, about 38 mm, about 39 mm, about 40 mm, about 41 mm, about 42 mm, about 43 mm, about 44 mm, about 45 mm, about 46 mm, about 47 mm, about 48 mm, about 49 mm, or about 50 mm). In some embodiments, the sensors are configured to measure tissue-borne sound. In some embodiments, the first and/or second plurality of external sensors are electrically connected to a sound recorder. In some embodiments, the sensors do not make direct contact with the subject (e.g., a plurality of sensors positioned at a bedside of the subject). In some embodiments, one or more primary external sensors of the first plurality of external sensors are configured to be removably secured to a plurality of positions of a face, head, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained. In some embodiments, the first plurality and or second plurality of external sensors are accelerometers. In some embodiments, the accelerometers are removably secured to the face, head, neck, and/or upper torso of the subject. In some embodiments, the accelerometers detect tissue vibrations caused by tissue-borne sounds (e.g., when a subject articulates a calibration sound). In some embodiments, the accelerometers are configured to collect sound at an amplitude of from about 30 dB to about 90 dB (e.g., about 30 dB, about 31 dB, about 32 dB, about 33 dB, about 34 dB, about 35 dB, about 36 dB, about 37 dB, about 38 dB, about 39 dB, about 40 dB, about 41 dB, about 42 dB, about 43 dB, about 44 dB, about 45 dB, about 46 dB, about 47 dB, about 48 dB, about 49 dB, about 50 dB, about 51 dB, about 52 dB, about 53 dB, about 54 dB, about 55 dB, about 56 dB, about 57 dB, about 58 dB, about 59 dB, about 60 dB, about 61 dB, about 62 dB, about 63 dB, about 64 dB, about 65 dB, about 66 dB, about 67 dB, about 68 dB, about 69 dB, about 70 dB, about 71 dB, about 72 dB, about 73 dB, about 74 dB, about 75 dB, about 76 dB, about 77 dB, about 78 dB, about 79 dB, about 80 dB, about 81 dB, about 82 dB, about 83 dB, about 84 dB, about 85 dB, about 86 dB, about 87 dB, about 88 dB, about 89 dB, or about 90 dB) and at a frequency range from about 20 Hz to about 20 kHz (e.g., from about 40 Hz to about 20 kHz, from about 60 Hz to about 15 kHz, from about 80 Hz to about 10 kHz, or from about 100 Hz to about 5 kHz). In some embodiments, the accelerometers have a sensitivity of from about 10 m V/g to about 500 m V/g (e.g., about 10 m V/g, about 11 mV/g, about 12 m V/g, about 13 mV/g, about 14 m V/g, about 15 m V/g, about 16 m V/g, about 17 m V/g, about 18 mV/g, about 19 mV/g, about 20 mV/g, about 21 m V/g, about 22 m V/g, about 23 m V/g, about 24 m V/g, about 25 m V/g, about 26 m V/g, about 27 m V/g, about 28 m V/g, about 29 m V/g, about 30 m V/g, about 31 m V/g, about 32 m V/g, about 33 m V/g, about 34 m V/g, about 35 m V/g, about 36 m V/g, about 37 m V/g, about 38 m V/g, about 39 m V/g, about 40 m V/g, about 41 m V/g, about 42 m V/g, about 43 m V/g, about 44 m V/g, about 45 m V/g, about 46 m V/g, about 47 m V/g, about 48 m V/g, about 49 m V/g, about 50 m V/g, about 51 m V/g, about 52 m V/g, about 53 m V/g, about 54 m V/g, about 55 m V/g, about 56 m V/g, about 57 m V/g, about 58 m V/g, about 59 m V/g, about 60 m V/g, about 61 m V/g, about 62 m V/g, about 63 m V/g, about 64 m V/g, about 65 m V/g, about 66 m V/g, about 67 m V/g, about 68 m V/g, about 69 m V/g, about 70 m V/g, about 71 mV/g, about 72 m V/g, about 73 m V/g, about 74 m V/g, about 75 m V/g, about 76 m V/g, about 77 m V/g, about 78 m V/g, about 79 m V/g, about 80 m V/g, about 81 mV/g, about 82 m V/g, about 83 m V/g, about 84 m V/g, about 85 m V/g, about 86 m V/g, about 87 m V/g, about 88 mV/g, about 89 m V/g, about 90 m V/g, about 91 m V/g, about 92 m V/g, about 93 m V/g, about 94 m V/g, about 95 m V/g, about 96 m V/g, about 97 m V/g, about 98 mV/g, about 99 m V/g, about 100 m V/g, about 110 m V/g, about 120 m V/g, about 130 m V/g, about 140 m V/g, about 150 m V/g, about 160 m V/g, about 170 m V/g, about 180 m V/g, about 190 m V/g, about 200 m V/g, about 210 m V/g, about 220 m V/g, about 230 m V/g, about 240 m V/g, about 250 m V/g, about 260 m V/g, about 270 m V/g, about 280 m V/g, about 290 m V/g, about 300 m V/g, about 310 m V/g, about 320 m V/g, about 330 m V/g, about 340 m V/g, about 350 m V/g, about 360 m V/g, about 370 m V/g, about 380 m V/g, about 390 m V/g, about 400 m V/g, about 410 m V/g, about 420 m V/g, about 430 m V/g, about 440 m V/g, about 450 m V/g, about 460 mV/g, about 470 m V/g, about 480 m V/g, about 490 mV/g, or about 500 m V/g). In some embodiments, the accelerometers have a dynamic range of from about 10 dB to about 100 dB (e.g., about 10 dB, about 11 dB, about 12 dB, about 13 dB, about 14 dB, about 15 dB, about 16 dB, about 17 dB, about 18 dB, about 19 dB, about 20 dB, about 21 dB, about 22 dB, about 23 dB, about 24 dB, about 25 dB, about 26 dB, about 27 dB, about 28 dB, about 29 dB, about 30 dB, about 31 dB, about 32 dB, about 33 dB, about 34 dB, about 35 dB, about 36 dB, about 37 dB, about 38 dB, about 39 dB, about 40 dB, about 41 dB, about 42 dB, about 43 dB, about 44 dB, about 45 dB, about 46 dB, about 47 dB, about 48 dB, about 49 dB, about 50 dB, about 51 dB, about 52 dB, about 53 dB, about 54 dB, about 55 dB, about 56 dB, about 57 dB, about 58 dB, about 59 dB, about 60 dB, about 61 dB, about 62 dB, about 63 dB, about 64 dB, about 65 dB, about 66 dB, about 67 dB, about 68 dB, about 69 dB, about 70 dB, about 71 dB, about 72 dB, about 73 dB, about 74 dB, about 75 dB, about 76 dB, about 77 dB, about 78 dB, about 79 dB, about 80 dB, about 81 dB, about 82 dB, about 83 dB, about 84 dB, about 85 dB, about 86 dB, about 87 dB, about 88 dB, about 89 dB, about 90 dB, about 91 dB, about 92 dB, about 93 dB, about 94 dB, about 95 dB, about 96 dB, about 97 dB, about 98 dB, about 99 dB, or about 100 dB).

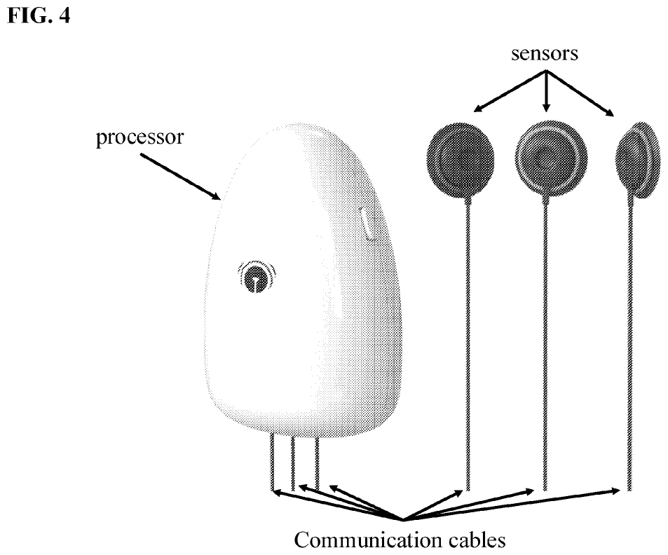

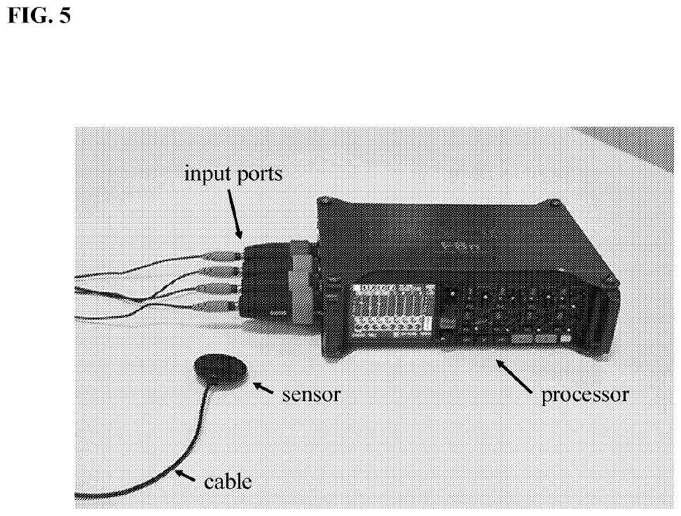

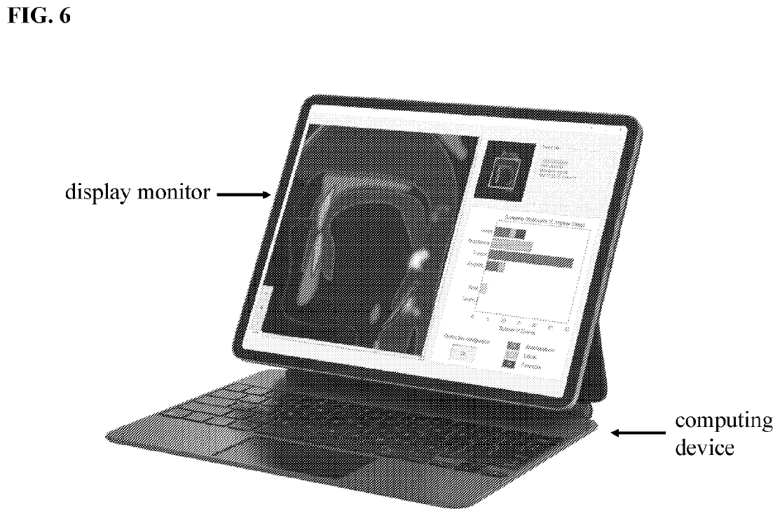

In some embodiments, the sensors are operably connected to a processor. In some embodiments, the sensors transmit tissue-borne sound data to the processor via a wire or cable (FIG. 4). In some embodiments, the processor includes a sound recorder. In some embodiments, the sound recorder has a sampling rate of from 44.1 kHz to 192 kHz (e.g., 44.1 kHz, 48 kHz, 88.2 kHz, 96 kHz, 176.4 kHz, or 192 kHz). In some embodiments, the sampling rate is 192 kHz and the bit depth is 16-or 24-bit. In some embodiments, the sensors wirelessly transmit tissue-borne sound data to the processor. In some embodiments, the processor includes a memory unit and a plurality of signal input (FIG. 5) and output ports. In some embodiments, the system includes a computing device. In some embodiments, the computing device is operably connected to a display monitor (FIG. 6).

In some embodiments, the memory unit of the processor is a configured to store one or more set of instructions. In some embodiments, the processor is electrically coupled to the plurality of input/output signal ports, the memory unit, and to a computing device (FIG. 6). The processor is configured to read a set of instructions stored in the memory unit order to 1) obtain a vocalization dataset via the plurality of sensors, wherein obtaining the vocalization dataset is based on the subject articulating a plurality of calibration sounds, 2) map the vocalization dataset to one or more locations of one or more anatomical areas of the subject, 3) obtain an airway dataset via the plurality of sensors removably secured to the subject, 4) identify a breathing event based on the airway dataset, and 5) correlate the breathing event to a location of the corresponding one or more locations. In some embodiments, the set of instructions, when executed by the processor, cause the system to display the airway dataset, a breathing event, a mapped breathing event, and a plurality of breathing metrics of the subject, on a digital display monitor and to transmit the airway dataset to a machine learning program.

In some embodiments, the computing device of the system includes a machine learning program configured to classify the breathing event, identified and correlated to a location of the corresponding one or more locations by the system, as a normal breathing event or an abnormal breathing event. In some embodiments, the machine learning program includes a plurality of algorithms configured to compare a training dataset to the airway dataset to classify one or more breathing events as normal or abnormal breathing events. In general, the system of the invention is configured to execute the method herein disclosed for detecting, localizing, and classifying a breathing event.

### Computer Implementation

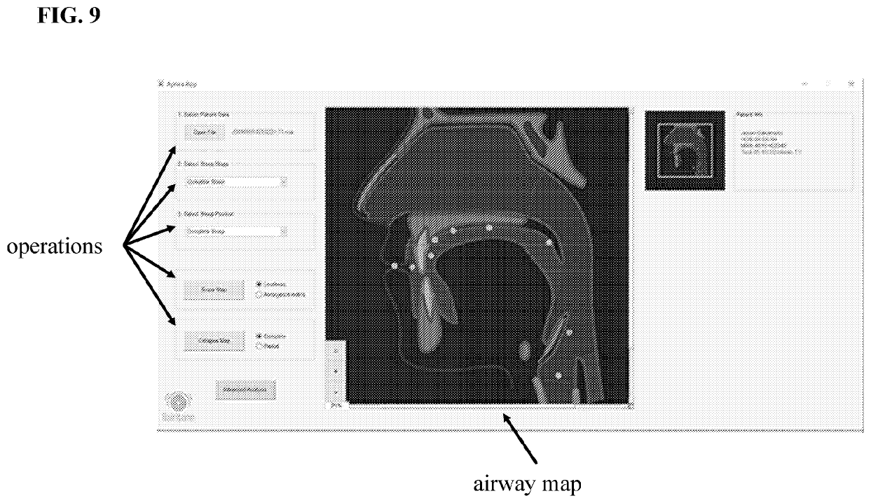

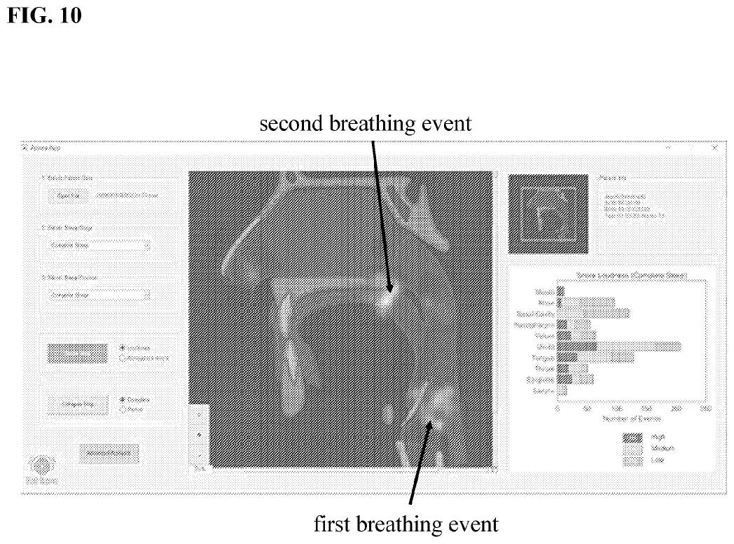

The methods described herein, including the methods of implementing one or more decision engines for detecting, localizing, processing, classifying, and reporting a breathing event (FIGS. 9-11), are, in some embodiments, performed on one or more computers.

For example, the building and deployment of any method described herein can be implemented in hardware or software, or a combination of both. In one embodiment, a machine-readable storage medium is provided, the medium comprising a data storage material encoded with machine readable data which, when using a machine programmed with instructions for using said data, is capable of executing any one of the methods described herein and/or displaying any of the datasets or results (e.g., breathing event detection, classification prediction) described herein. Some embodiments can be implemented in computer programs executing on programmable computers, comprising a processor and a data storage system (including volatile and non-volatile memory and/or storage elements), and optionally including a graphics adapter, a pointing device, a network adapter, at least one input device, and/or at least one output device. A display may be coupled to the graphics adapter. Program code is applied to input data to perform the functions described above and generate output information. The output information is applied to one or more output devices, in known fashion. The computer can be, for example, a personal computer, microcomputer, workstation, smartphone, or tablet of conventional design.

Each program can be implemented in a high-level procedural or object-oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of an embodiment. The databases of some embodiments can be recorded on computer readable media, e.g., any medium that can be read and accessed directly by a computer. Such media include, but are not limited to magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

In some embodiments, the methods described herein, including the methods for detecting, localizing, processing, classifying, and reporting a breathing event, are performed on one or more computers in a distributed computing system environment (e.g., in a cloud computing environment). In this description, “cloud computing” is defined as a model for enabling on-demand network access to a shared set of configurable computing resources. Cloud computing can be employed to offer on-demand access to the shared set of configurable computing resources. The shared set of configurable computing resources can be rapidly provisioned via virtualization and released with low management effort or service provider interaction, and then scaled accordingly. A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), Testing as a Service (“TaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

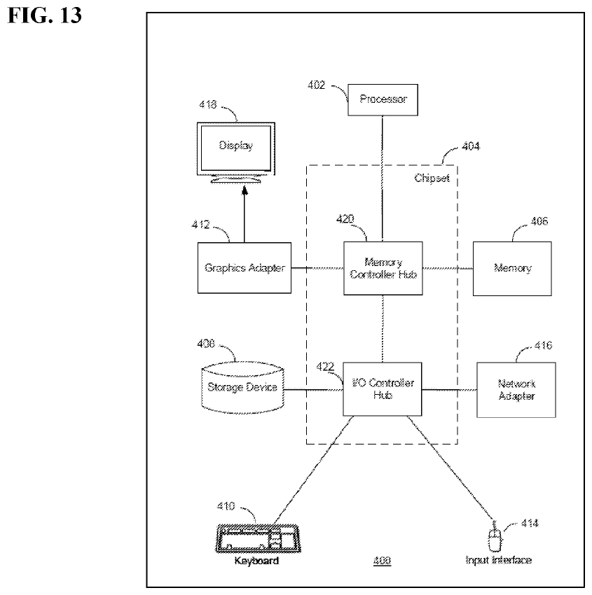

FIG. 13 illustrates an example computer for implementing the entities shown in FIGS. 1, 12, and 14. The computer 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input device 414, and network adapter 416 are coupled to the I/O controller hub 422. Other embodiments of the computer 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of pointing device, a keyboard, or some combination thereof, and is used to input data into the computer 400. In some embodiments, the computer 400 may be configured to receive input (e.g., commands) from the input interface 414 via gestures from the user. The network adapter 416 couples the computer 400 to one or more computer networks.

The graphics adapter 412 displays images and other information on the display 418. In various embodiments, the display 418 is configured such that the user may (e.g., subject, healthcare professional, non-healthcare professional) may input user selections on the display 418 to, for example, initiate the system for detecting, localizing, processing, classifying, and reporting a breathing event. In one embodiment, the display 418 may include a touch interface. In various embodiments, the display 418 can show a cardiac health status for the subject and associated monitoring. Thus, a user who accesses the display 418 can inform the subject of the cardiac health status. In various embodiments, the display 418 can show information such as depicted in FIGS. 6 and 9-11.

The computer 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computers 400 used can vary depending upon the embodiment and the processing power required by the method. For example, the step 1402 of FIG. 14 can run in a single computer 400 or multiple computers 400 communicating with each other through a network such as in a server farm. The computers 400 can lack some of the components described above, such as graphics adapters 412, and displays 418.

### EXAMPLES

The examples presented herein represent certain embodiments of the present disclosure. However, it is to be understood that these examples are for illustration purposes only and do not intend, nor should any be construed, to be wholly definitive as to conditions and scope of this invention. The examples were carried out using standard techniques, which are well known and routine to those of skill in the art, except where otherwise described in detail. Any of the above aspects and embodiments can be combined with any other aspect or embodiment as disclosed in the Drawings, in the Summary of the Invention, and/or in the Detailed Description, including the below Examples.

### Example 1. Diagnosing a Patient With Sleep Apnea and Determining the Location of the Apneic Event

A patient experiencing trouble sleeping attends a sleep study center for an evaluation of her airway. A sleep study specialist secures eight sensors on the patient's face, three on the right side of the patient's face, three on the left side of the patient's face, and one on each side of the patient's neck near the patient's throat. The sensors are connected via a cable to the processor and the processor is connected via another cable to a computing device. The computing device is a laptop computer having a display monitor. After securing the sensors to the patient's face and neck, the sleep study specialist provides the patient a series of words to read and vocalize. The specialist records the vocalization dataset of the patient and stores the data in the processor. Then the specialist opens a software application on the laptop computer, imports the patient's vocalization dataset to the software application, and instructs the software program to execute a process to map the vocalization dataset to the airway of the subject. One of the sensors is designated as the reference sensor to determine arrival time difference and amplitude difference of the sounds in the vocalization dataset in relation to the other sensors. The software program obtains amplitude and time difference data for all sounds produced by the patient when vocalizing the series of words and compares the amplitude and time difference data of the vocalization dataset to the amplitude and time difference recorded by the reference sensor. Having mapped the vocalization dataset to the patient's airway, the specialist instructs the patient to fall asleep. The sensors are maintained in the same position during the sleep portion of the study. As the patient sleeps the software records the airway dataset and captures all tissue-borne sounds in the airway of the patient. After the patient awakens, the recording of the airway dataset is finished and saved to processor's memory unit or the laptop's memory. The specialist then imports the airway dataset into the software application. Then the specialist instructs the software program to perform a machine learning based analysis of the airway dataset to identify any acoustic signatures that are characteristic to a breathing event. In the airway dataset, during a 40 second sample of the airway data, normal breathing sound, an apnea region, and an acoustic signature for the reopening of the collapsed airway is observed. During the apnea region, various signatures representative of choking are recorded. The machine learning program identifies the number of and duration of apneic and hypoapneic events. The machine learning program identifies an apneic event in the oropharynx of the patient and classifies it as such. The machine learning program also identifies snoring, normal breathing, and coughing events throughout the airway dataset. A report and a graphical representation of the airway dataset is generated (FIGS. 9-11) and presented to the sleep study specialist for treatment determination.

### Example 2. Diagnosing a Patient With a Partial Airway Collapse and Determining the Location and the Configuration of the Partial Airway Collapse

A patient experiencing trouble sleeping attends a sleep study center for an evaluation of his airway. A sleep study specialist secures six sensors on the patient's face, two on the right side of the patient's face, two on the left side of the patient's face, and one on each side of the patient's neck near the patient's throat. The sensors are connected via a cable to the processor and the processor is connected via another cable to a computing device. The computing device is a laptop computer having a display monitor. After securing the sensors to the patient's face and neck, the sleep study specialist provides the patient a series of words to read and vocalize. The specialist records the vocalization dataset of the patient and stores the data in the processor. Then the specialist opens a software application on the laptop computer, imports the patient's vocalization dataset to the software application, and instructs the software program to execute a process to map the vocalization dataset to the airway of the subject. One of the sensors is designated as the reference sensor to determine arrival time difference and amplitude difference of the sounds in the vocalization dataset in relation to the other sensors. The software program obtains amplitude and time difference data for all sounds produced by the patient when vocalizing the series of words and compares the amplitude and time difference data of the vocalization dataset to the amplitude and time difference recorded by the reference sensor. Having mapped the vocalization dataset to the patient's airway, the specialist instructs the patient to fall asleep. The sensors are maintained in the same position during the sleep portion of the study. As the patient sleeps the software records the airway dataset and captures all tissue-borne sounds in the airway of the patient. After the patient awakens, the recording of the airway dataset is finished and saved to processor's memory unit or the laptop's memory. The specialist then imports the airway dataset into the software application. Then the specialist instructs the software program to perform a machine learning based analysis of the airway dataset to identify any acoustic signatures that are characteristic to a breathing event. The machine learning program identifies a partial collapse event in the hypopharynx of the patient and classifies it as such. The machine learning program also determines that the partial collapse event is a lateral event where the left side and the right side of the hypopharynx partially collapsed during the event. The machine learning program also identifies snoring, normal breathing, and coughing events throughout the airway dataset. A report and a graphical representation of the airway dataset is generated and presented to the sleep study specialist for treatment determination.

### Example 3. Diagnosing a Patient With Multiple Airway Events and Determining the Location and the Configuration of Each of the Airway Events

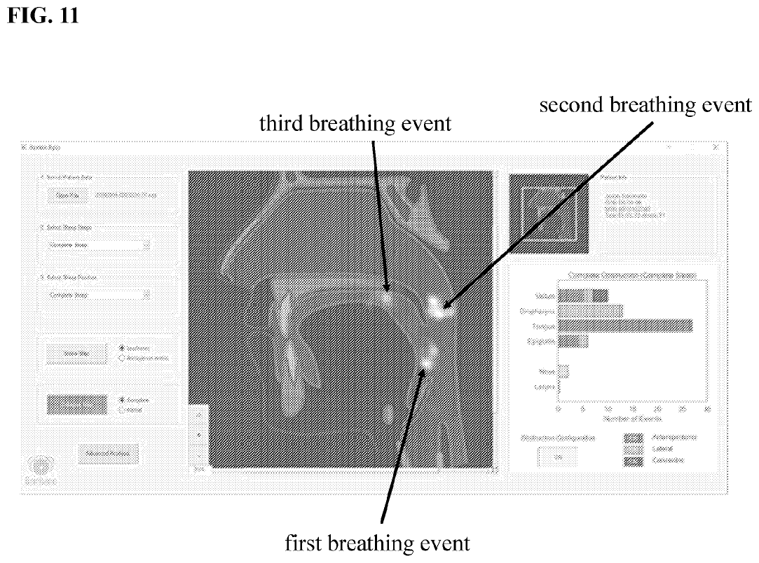

A patient experiencing trouble sleeping attends a sleep study center for an evaluation of her airway. A sleep study specialist secures ten sensors on the patient's face, four on the right side of the patient's face, four on the left side of the patient's face, and one on each side of the patient's neck near the patient's throat. The sensors are connected via a cable to the processor and the processor is connected via another cable to a computing device. The computing device is a laptop computer having a display monitor. After securing the sensors to the patient's face and neck, the sleep study specialist provides the patient a series of words to read and vocalize. The specialist records the vocalization dataset of the patient and stores the data in the processor. Then the specialist opens a software application on the laptop computer, imports the patient's vocalization dataset to the software application, and instructs the software program to execute a process to map the vocalization dataset to the airway of the subject. One of the sensors is designated as the reference sensor to determine arrival time difference and amplitude difference of the sounds in the vocalization dataset in relation to the other sensors. The software program obtains amplitude and time difference data for all sounds produced by the patient when vocalizing the series of words and compares the amplitude and time difference data of the vocalization dataset to the amplitude and time difference recorded by the reference sensor. Having mapped the vocalization dataset to the patient's airway, the specialist instructs the patient to fall asleep. The sensors are maintained in the same position during the sleep portion of the study. As the patient sleeps the software records the airway dataset and captures all tissue-borne sounds in the airway of the patient. After the patient awakens, the recording of the airway dataset is finished and saved to processor's memory unit or the laptop's memory. The specialist then imports the airway dataset into the software application and divides airway dataset into intervals of 30 minutes each. Then the specialist instructs the software program to perform a machine learning based analysis of the airway dataset to identify any acoustic signatures that are characteristic to a breathing event. The machine learning program identifies a partial collapse event in the hypopharynx of the patient and a full collapse in the velum region of the patient's airway (FIG. 11). The machine learning program classifies that the partial collapse event is a lateral event where the left side and the right side of the hypopharynx partially collapsed during the event. The machine learning program classifies the full collapse near the velum as a concentric collapse. The machine learning program also identifies snoring, normal breathing, and coughing events throughout the airway dataset. A report and a graphical representation of the airway dataset is generated and presented to the sleep study specialist for treatment determination.

### Example 4. Identifying a Patient Who May be Contraindicated for Hypoglossal Nerve Implant Surgery

A patient experiencing trouble sleeping attends a sleep study center for an evaluation of her airway. A sleep study specialist secures ten sensors on the patient's face, four on the right side of the patient's face, four on the left side of the patient's face, and one on each side of the patient's neck near the patient's throat. The sensors are connected via a cable to the processor and the processor is connected via another cable to a computing device. The computing device is a laptop computer having a display monitor. After securing the sensors to the patient's face and neck, the sleep study specialist provides the patient a series of words to read and vocalize. The specialist records the vocalization dataset of the patient and stores the data in the processor. Then the specialist opens a software application on the laptop computer, imports the patient's vocalization dataset to the software application, and instructs the software program to execute a process to map the vocalization dataset to the airway of the subject. One of the sensors is designated as the reference sensor to determine arrival time difference and amplitude difference of the sounds in the vocalization dataset in relation to the other sensors. The software program obtains amplitude and time difference data for all sounds produced by the patient when vocalizing the series of words and compares the amplitude and time difference data of the vocalization dataset to the amplitude and time difference recorded by the reference sensor. Having mapped the vocalization dataset to the patient's airway, the specialist instructs the patient to fall asleep. The sensors are maintained in the same position during the sleep portion of the study. As the patient sleeps the software records the airway dataset and captures all tissue-borne sounds in the airway of the patient. After the patient awakens, the recording of the airway dataset is finished and saved to processor's memory unit or the laptop's memory. The specialist then imports the airway dataset into the software application. Then the specialist instructs the software program to perform a machine learning based analysis of the airway dataset to identify any acoustic signatures that are characteristic to a breathing event. The machine learning program identifies a number of instances during the patient's sleep that the patient's breathing stopped. However, the airway dataset did not contain any data to suggest at least a partial collapse or obstruction of the patient's airway. Thus, the machine learning program classifies that the patient experienced central sleep apnea. The machine learning program also identifies snoring, normal breathing, and coughing events throughout the airway dataset. A report and a graphical representation of the airway dataset is generated and presented to the sleep study specialist for treatment determination. The patient is deemed contraindicated for hypoglossal nerve stimulation implant surgery.

### Example 5. Identifying a Patient Who Has Epiglottic Trap Door Phenomenon

A patient experiencing trouble sleeping attends a sleep study center for an evaluation of her airway. A sleep study specialist secures ten sensors on the patient's face, four on the right side of the patient's face, four on the left side of the patient's face, and one on each side of the patient's neck near the patient's throat. The sensors are connected via a cable to the processor and the processor is connected via another cable to a computing device. The computing device is a laptop computer having a display monitor. After securing the sensors to the patient's face and neck, the sleep study specialist provides the patient a series of words to read and vocalize. The specialist records the vocalization dataset of the patient and stores the data in the processor. Then the specialist opens a software application on the laptop computer, imports the patient's vocalization dataset to the software application, and instructs the software program to execute a process to map the vocalization dataset to the airway of the subject. One of the sensors is designated as the reference sensor to determine arrival time difference and amplitude difference of the sounds in the vocalization dataset in relation to the other sensors. The software program obtains amplitude and time difference data for all sounds produced by the patient when vocalizing the series of words and compares the amplitude and time difference data of the vocalization dataset to the amplitude and time difference recorded by the reference sensor. Having mapped the vocalization dataset to the patient's airway, the specialist instructs the patient to fall asleep. The sensors are maintained in the same position during the sleep portion of the study. As the patient sleeps the software records the airway dataset and captures all tissue-borne sounds in the airway of the patient. After the patient awakens, the recording of the airway dataset is finished and saved to processor's memory unit or the laptop's memory. The specialist then imports the airway dataset into the software application. Then the specialist instructs the software program to perform a machine learning based analysis of the airway dataset to identify any acoustic signatures that are characteristic to a breathing event. The machine learning program identified acoustic signatures characteristic of floppy closing door epiglottis. The machine learning program also identifies snoring, normal breathing, and coughing events throughout the airway dataset. A report and a graphical representation of the airway dataset is generated and presented to the sleep study specialist for treatment determination.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In addition, the materials, methods, and examples are illustrative only and are not intended to be limiting.

What is claimed is:

1. A method of analyzing an airway of a subject comprising:
a) obtaining a vocalization dataset via a first plurality of external sensors based on the subject articulating a plurality of calibration sounds;
b) generating a mapped vocalization dataset by mapping the vocalization dataset to the airway of the subject or a portion thereof;
c) obtaining an airway dataset of the airway of the subject via the first plurality of external sensors and/or a second plurality of external sensors;
d) identifying a breathing event based on the airway dataset; and
e) localizing the breathing event to one or more locations of the airway or the portion thereof using the airway dataset and the mapped vocalization dataset;
wherein steps a) through e) are performed in any order of sequence.

2. The method of claim 1, further comprising:
a) removably securing one or more primary external sensors of the first plurality of external sensors to the head, face, neck, and/or upper torso of the subject prior to obtaining the vocalization dataset; and/or
b) positioning one or more secondary external sensors of the first plurality of external sensors at an optimal distance from the head, face, neck, and/or upper torso of the subject prior to obtaining the vocalization dataset;
wherein the one or more secondary external sensors are not in direct contact with the subject.

3. The method of claim 2, wherein the optimal distance is from about 1 cm to about 500 cm.

4. The method of any one of claims 1-3, wherein the vocalization dataset comprises tissue-borne sound data of a plurality of frequency bands for each calibration sound articulated by the subject.

5. The method of any one of claims 1-4, wherein the calibration sounds comprise speech-based consonant sounds and/or non-speech-based consonant-like sounds produced by the mouth and/or airway of the subject.

6. The method of any one of claims 1-5, wherein mapping the vocalization dataset comprises measuring i) an amplitude difference and ii) a time difference corresponding to each calibration sound and wherein i) and ii) are measured by one or more of the external sensors in relation to a reference sensor.

7. The method of claim 6, further comprising correlating each amplitude difference and time difference measurement to a location of the one or more locations, based on the corresponding calibration sound, so as to generate a calibration dataset.

8. The method of any one of claims 1-7, wherein localizing the breathing event comprises:
a) obtaining, with the one or more plurality of external sensors, i) an amplitude difference measurement and ii) a time difference measurement for the breathing event; and
b) comparing the amplitude difference measurement and the time difference measurement for the breathing event to the amplitude difference measurement and the time difference measurement of the calibration dataset;
wherein i) and ii) are measured in relation to the reference sensor for a prescribed duration.

9. The method of claim 8, wherein the prescribed duration is a time interval of from about 10 seconds to 1 hour.

10. The method of any one of claims 1-9, wherein the breathing event is produced by the subject during sleep.

11. The method of any one of claims 1-10, wherein the breathing event comprises an airway collapse event, a partial airway collapse event, a sleep apnea event, an apneic event, a hypoapneic event, a snore event, an upper airway occlusion event, a cessation of breathing, a respiratory disturbance event, ventilatory instability, normal breathing, a change in airflow, or any combination thereof.

12. The method of any one of claims 1-11, wherein the one or more locations of the airway or the portion thereof comprises a velum, an oropharynx, a hypopharynx, a tongue, and/or an epiglottis.

13. The method of any one of claims 1-12, wherein the at least one plurality of external sensors comprises of from 2 to 10 external sensors.

14. The method of any one of claims 1-13, further comprising removably securing the first plurality of external sensors on opposite sides of the subject's head or neck.

15. The method of any one of claims 1-14, further comprising removably securing the first plurality of external sensors on the same side of the subject's head or neck.

16. The method of any one of claims 1-15, wherein each of the first and/or second plurality of external sensors comprises a microphone and/or an accelerometer.

17. The method of any one of claims 1-16, wherein each of the first and/or second plurality of external sensors further comprises a sound emitting unit.

18. The method of any one of claims 1-17, wherein the vocalization dataset is obtained while the subject is awake.

19. The method of any one of claims 1-18, wherein the first plurality of external sensors are located at a first plurality of positions of a head, face, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained.

20. The method of any one of claims 1-19, wherein the airway dataset further comprises one or more sounds associated with an airway condition comprising anaphylaxis, sleep apnea, upper airway resistance syndrome, throat cancer, head cancer, neck cancer, chronic obstructive pulmonary disease, stridor, speech language disorders, a speech impediment, an accent, an infection, inflammation, an airway narrowing, or a laryngeal web.

21. The method of any one of claims 1-20, further comprising:
a) teaching a machine learning software to identify a breathing event and/or classify a breathing event using a reference dataset; and
b) presenting the airway dataset comprising one or more breathing events to the machine learning software;
wherein the machine learning software compares the one or more breathing events to the reference dataset to identify the breathing event and/or classify the breathing event as a normal or abnormal breathing event.

22. The method of claim 21, further comprising classifying the breathing event as a lateral collapse, an anterior-to-posterior collapse, or a concentric collapse.

23. The method of any one of claims 1-22, wherein obtaining the airway dataset comprises determining the location of one or more sounds in the airway using beamforming or reference mapping.

24. The method of any one of claims 1-23, wherein the first plurality of external sensors is removably secured to the forehead of the subject, a temple of the subject, a cheekbone of the subject, adjacent to a nose of the subject, a mastoid of the subject, a mandible of the subject, and/or a throat of the subject.

25. The method of claim 4, wherein the plurality of frequency bands span a frequency range of from about 20 Hz to about 20 kHz.

26. The method of claim 4, wherein the plurality of frequency bands span a frequency range of from about 100 Hz to about 5 kHz.

27. The method of claim 4, wherein the plurality of frequency bands comprises from 2 to 1,000 frequency bands.

28. The method of claim 4, wherein each frequency band comprises a bandwidth of from about 5 Hz to about 2500 Hz.

29. The method of any one of claims 1-28, further comprising determining one or more metric(s) including Apnea-Hypopnea Index (AHI), blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, and/or actigraphy.

30. An airway analysis system comprising:
a) a first plurality of external sensors;
b) a processor in operative communication with the first plurality of external sensors; and
c) a memory unit storing instructions that, when executed by the processor, cause the system to perform a plurality of operations including:
i. obtaining a vocalization dataset via the first plurality of external sensors, based on the subject articulating a plurality of calibration sounds;
ii. generating a mapped vocalization dataset by mapping the vocalization dataset to the airway of the subject or a portion thereof;
iii. obtaining an airway dataset of the airway of the subject via the first and/or a second plurality of external sensors;
iv. identifying a breathing event based on the airway dataset; and
v. localizing the breathing event to one or more locations of the airway or the portion thereof using the airway dataset and the mapped vocalization dataset;
wherein steps i) through v) are performed in any order of sequence.

31. The system of claim 30, further comprising:
a) one or more primary external sensors of the first plurality of external sensors configured to be removably secured to the head, face, neck, and/or upper torso of the subject; and/or
b) one or more secondary external sensors of the first plurality of external sensors configured to be positioned at an optimal distance from the head, face, neck, and/or upper torso of the subject;
wherein the one or more secondary external sensors are configured to not directly contact the subject.

32. The system of claim 31, wherein the optimal distance is from about 1 cm to about 500 cm.

33. The system of any one of claims 30-32, wherein the first plurality of external sensors is configured to measure tissue-borne sound for a given location of an anatomical area of the subject when the subject articulates a calibration sound.

34. The system of any one of claims 30-33, further comprising a computing device.

35. The system of claim 34, wherein the computing device comprises a machine learning program configured to identify and/or classify the breathing event, and correlate the breathing event to a location of the corresponding one or more locations of the airway of subject, as a normal breathing event or an abnormal breathing event.

36. The system of any one of claims 30-35, wherein the first plurality of external sensors comprises from 2 to 10 external sensors.

37. The system of claim 36, wherein each of the first and/or second plurality of external sensors comprises a microphone and/or an accelerometer.

38. The system of 36, wherein each of the first and/or second plurality of external sensors further comprises a sound emitting unit.

39. The system of any one of claims 30-38, wherein the first and/or second plurality of external sensors are electrically connected to a sound recorder.

40. The system of any one of claims 30-39, wherein the instructions, when executed by the processor, cause the system to perform operations further including displaying the airway dataset on a digital display monitor and transmitting the airway dataset to a machine learning program.

41. The system of claim 40, wherein the machine learning program comprises a plurality of algorithms configured to compare a training dataset to the airway dataset to classify one or more breathing events as normal or abnormal breathing events.

42. The system of any one of claims 30-41, wherein the vocalization dataset comprises tissue-borne sound data of a plurality of frequency bands for each calibration sound articulated by the subject.

43. The system of any one of claims 30-42, wherein the calibration sounds comprise speech-based consonant sounds and/or non-speech-based consonant-like sounds produced by the mouth and/or airway of the subject.

44. The system of any one of claims 30-43, wherein the operation of mapping the vocalization dataset comprises measuring i) an amplitude difference and ii) a time difference corresponding to each calibration sound and wherein i) and ii) are measured by one or more external sensors of the at least one plurality of external sensors in relation to a reference sensor.

45. The system of claim 44, further configured to correlate each amplitude difference and time difference measurement to a location of the one or more locations, based on the corresponding calibration sound, so as to generate a calibration dataset.

46. The system of any one of claims 30-45, wherein the breathing event is produced by the subject during sleep.

47. The system of claim 46, wherein the breathing event comprises an airway collapse event, a partial airway collapse event, an apneic event, a hypoapneic event, a snore event, an upper airway occlusion event, a cessation of breathing, a respiratory disturbance event, ventilatory instability, normal breathing, a change in airflow, or any combination thereof.

48. The system of any one of claims 30-47, wherein localizing the breathing event to the one or more locations of the airway of the subject, or the portion thereof, comprises:
a) obtaining, with the first and/or second plurality of external sensors, i) an amplitude difference measurement and ii) a time difference measurement for the breathing event; and
b) comparing i) and ii) to the calibration dataset;
wherein i) and ii) are measured in relation to a reference sensor for a prescribed duration.

49. The system of claim 48, wherein the prescribed duration is a time interval of from about 10 seconds to 1 hour.

50. The system of any one of claims 30-49, wherein the one or more locations of the airway of the subject, or the portion thereof, comprises a velum, an oropharynx, a tongue, and/or an epiglottis.

51. The system of any one of claims 30-50, wherein the first plurality of external sensors are located at a first plurality of positions of a head, neck, and/or upper torso of the subject, and wherein obtaining the airway dataset is performed by the first and/or second plurality of external sensors located at substantially the same plurality of positions of the head, face, neck, and/or upper torso of the subject from where the vocalization dataset is obtained.

52. The system of any one of claims 30-51, configured to determine one or more metric(s) including Apnea-Hypopnea Index (AHI), blood oxygenation, respiration rate, heart rate, electroencephalogram, electrooculogram, electromyogram, electrocardiogram, nasal and oral airflow, breathing and respiratory effort, pulse oximetry, arterial oxygen saturation, chest wall movement, abdominal wall movement, and/or actigraphy substantially in parallel to the plurality of operations.

53. A method of analyzing an airway of a subject comprising:
a) removably securing a plurality of external sensors to the subject;
b) recording a vocalization dataset;
c) recording an airway dataset; and
d) comparing the airway dataset to the vocalization dataset;
wherein recording the vocalization dataset comprises recording the subject articulating a plurality of calibration sounds.
